(19) United States
(12) Patent Application Publication
(10) Pub. No.: US 2026/0144973 A1
(43) Pub. Date: May 28, 2026

(54) DETERMINING PORT HEALTH WITH ULTRASOUND

(71) Applicant: FUJIFILM SONOSITE, INC., Bothell, WA (US)

(72) Inventors: Craig Chamberlain, Seattle, WA (US); Jimin Zhang, Bellevue, WA (US); Katsuya Yamamoto, Kanagawa (JP); Marnie Hamp, Kirkland, WA (US); Keith Williams, Seattle, WA (US); Thomas Endres, Sagle, ID (US); Richard Kelly, Bothell, WA (US)

(21) Appl. No.: 18/957,403

(22) Filed: Nov. 22, 2024

(51) Int. Cl. A61M 39/02; G01S 7/52; G01S 7/539; G01S 15/89

(52) U.S. Cl. CPC A61M 39/0247; G01S 7/52058; G01S 7/539; G01S 15/8927; A61M 2205/3375; A61M 2205/502; A61M 2205/583

(57) ABSTRACT

Ultrasound systems, ultrasound scanners, and methods for determining port health using ultrasound are disclosed. In some embodiments, the ultrasound system includes a display device configured to display a user interface for the ultrasound system and a wearable ultrasound scanner configured to be attached to a patient over a port that is placed inside the patient, where the port is configured to supply fluid to the patient or retrieve additional fluid from the patient. The ultrasound system also includes a processor system configured to generate, based on reflections of ultrasound received by the wearable ultrasound scanner, a health status of the port, and cause the user interface to display the health status of the port.

### FIELD

Embodiments disclosed herein relate to ultrasound systems. More specifically, embodiments disclosed herein are related to determining port health using ultrasound.

### BACKGROUND

Many medical conditions require the repeated insertion and/or removal of fluid into a patient's body, such as the use of chemotherapy for cancer treatment, infection that requires long-term intravenous (IV) antibiotics, kidney failure that requires dialysis, inflammatory bowel disease (IBD) that requires parenteral IV nutrition, diseases that require multiple blood transfusions (e.g., liver disease, sickle-cell anemia, etc.), and the like. To reduce the impact on the patient anatomy that can be caused by the repeated use of a needle to insert and/or remove the fluid, the patient may be fitted with a port.

A port is an implantable reservoir with a tube attached to it that can be inserted into a blood vessel. The reservoir portion of the port is placed just beneath the patient's skin, and the tube can be inserted into the patient's blood vessel (e.g., vein). Fluid can then be inserted and/or removed by inserting a needle into the port, rather than directly into the blood vessel, thus eliminating painful needle sticks into the blood vessel, and the damage caused by the needle sticks. However, ports have high failure rates, typically up to 50%. Failed ports can prevent or delay a procedure, become a source of infection, and cause additional cost and patient harm. For instance, a failed port may need to be replaced, which can require the patient to undergo an additional surgery with anesthesia.

### SUMMARY

Ultrasound systems, ultrasound scanners, and methods for determining port health using ultrasound are disclosed. In some embodiments, the ultrasound system includes a display device configured to display a user interface for the ultrasound system and a wearable ultrasound scanner configured to be attached to a patient over a port that is placed inside the patient, where the port is configured to supply fluid to the patient or retrieve additional fluid from the patient. The ultrasound system also includes a processor system configured to generate, based on reflections of ultrasound received by the wearable ultrasound scanner, a health status of the port, and cause the user interface to display the health status of the port.

In some other embodiments, the ultrasound system includes a wearable ultrasound scanner configured to be attached to a patient over a port that is placed inside the patient, the port configured to supply fluid to the patient or retrieve additional fluid from the patient, where the wearable ultrasound scanner includes insertion holes through which a needle can be inserted into the port for the supply of the fluid or the retrieval of the additional fluid. The ultrasound system also includes a processor system configured to generate a health status of the port based on reflections of ultrasound received by the wearable ultrasound scanner, determine a recommended one of the insertion holes for the insertion of the needle based on the health status of the port, and cause an indication of the recommended one of the insertion holes to be exposed.

In yet some other embodiments, the ultrasound system includes a wearable ultrasound scanner and a display device configured to display guidance for placing the wearable ultrasound scanner over a port that is placed inside a patient, where the port is configured to supply fluid to the patient or retrieve additional fluid from the patient. The ultrasound system also includes a processor system configured to generate a health status of the port based on reflections of ultrasound received by the wearable ultrasound scanner, and generate the guidance for placement of the wearable ultrasound scanner based on the health status of the port.

Other aspects and advantages of the embodiments will become apparent from the following detailed description taken in conjunction with the accompanying drawings which illustrate, by way of example, the principles of the described embodiments.

### BRIEF DESCRIPTION OF THE DRAWINGS

The appended drawings illustrate examples and are, therefore, exemplary embodiments and not considered to be limiting in scope.

FIG. 1 illustrates some embodiments of an ultrasound system in a environment for determining port health with ultrasound during an ultrasound examination.

FIG. 2 illustrates some embodiments of an ultrasound system from FIG. 1.

FIG. 3 illustrates some embodiments of a system in an environment for determining port health with ultrasound.

FIG. 4 illustrates some embodiments of a system for determining port health with ultrasound.

FIG. 5 illustrates some embodiments of a user interface of a system for determining port health with ultrasound.

FIG. 6 illustrates some embodiments of example configurations of a reconfigurable wearable ultrasound scanner for determining port health with ultrasound.

FIG. 7 illustrates an environment with an example ultrasound scanner used for in-plane needle insertion.

FIG. 8 illustrates of an environment with an example ultrasound scanner used for out-of-plane needle insertion.

FIG. 9 illustrates some embodiments of an example multi-array transducer for determining port health with ultrasound.

FIG. 10 illustrates some embodiments of example characteristics of a multi-array transducer for determining port health with ultrasound.

FIG. 11 illustrates some embodiments of a multi-array transducer for determining port health with ultrasound.

FIG. 12 illustrates some embodiments of a multi-array transducer for determining port health with ultrasound.

FIG. 13 illustrates some embodiments of tuning impedances for transducer arrays for determining port health with ultrasound.

FIG. 14 illustrates some embodiments of array configurations for a multi-array transducer of an ultrasound scanner for determining port health with ultrasound.

FIG. 15 illustrates some embodiments of a machine-learning architecture used to train a machine-learned model.

FIG. 16 illustrates some embodiments of a machine-learned model using a CNN.

FIG. 17 illustrates some embodiments of an example computing device for determining port health with ultrasound.

FIG. 18 illustrates some embodiments of a method for determining port health with ultrasound.

FIG. 19 illustrates some other embodiments of a method for determining port health with ultrasound.

FIG. 20 illustrates yet some other embodiments of a method for determining port health with ultrasound.

### DETAILED DESCRIPTION

In the following description, numerous details are set forth to provide a more thorough explanation of the present invention. It will be apparent, however, to one skilled in the art, that the present invention may be practiced without these specific details. In other instances, well-known structures and devices are shown in block diagram form, rather than in detail, in order to avoid obscuring the present invention.

Monitoring of a port can be done with ultrasound. Ultrasound systems can generate ultrasound images by transmitting sound waves at frequencies above the audible spectrum into a body, receiving echo signals caused by the sound waves reflecting from internal body parts, and converting the echo signals into electrical signals for image generation. Ultrasound systems for port monitoring rely on a clinician (e.g., sonographer, nurse, doctor, or other trained operator) to acquire the ultrasound data. This monitoring can require repeated visits to a care facility or imaging facility and be inconvenient and expensive for the patient.

Accordingly, embodiments described herein include systems, devices, and methods for determining port health with ultrasound. In some embodiments, an ultrasound system includes a wearable ultrasound scanner that includes a patch configured for placement on a patient's skin over a port. The ultrasound system can implement one or more machine-learned models to generate a health status report that includes a prediction of when the port will fail. The wearable ultrasound scanner can include a multi-array transducer. In some embodiments, the multi-array transducer includes arrays comprised of lead zirconate titanate (PZT) array elements, capacitive micromachined ultrasonic transducer (CMUT) array elements, and/or piezoelectric micromachined ultrasonic transducer (PMUT) array elements. These and other aspects of determining port health with ultrasound are described in more detail below.

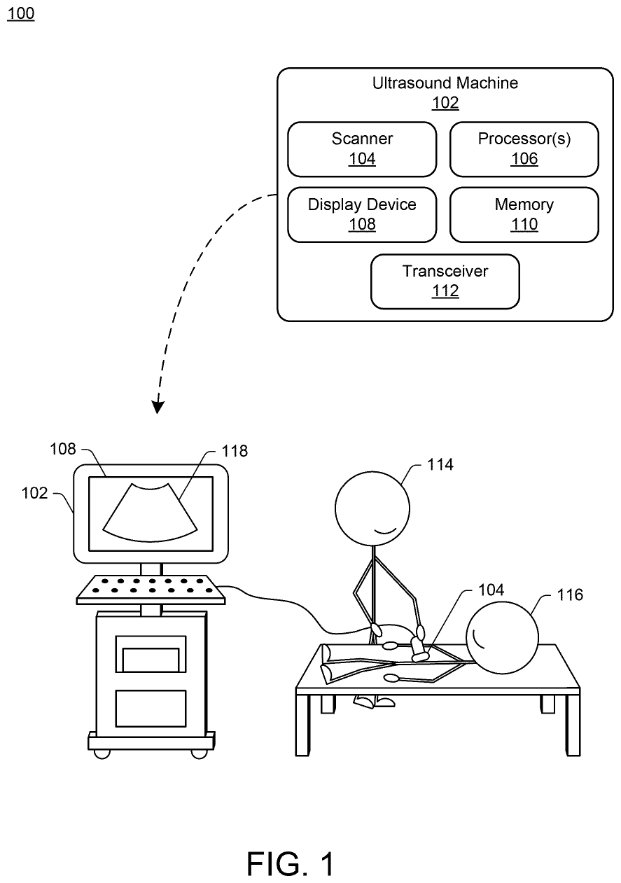

FIG. 1 illustrates an ultrasound system in an environment 100 for determining port health with ultrasound. Many medical conditions require the repeated insertion and/or removal of fluid into a patient's body, such as the use of chemotherapy for cancer treatment, infection that requires long-term intravenous (IV) antibiotics, kidney failure that requires dialysis, inflammatory bowel disease (IBD) that requires parenteral IV nutrition, diseases that require multiple blood transfusions (e.g., liver disease, sickle-cell anemia, etc.), and the like. To reduce the impact on the patient anatomy that can be caused by the repeated use of a needle to insert and/or remove the fluid, the patient may be fitted with a port.

The ultrasound system in FIG. 1 includes an ultrasound machine 102 and an ultrasound scanner 104. The ultrasound machine 102 generates high-frequency sound waves (e.g., ultrasound) and imaging data based on the ultrasound reflecting off a patient anatomy/body structure and/or an interventional instrument (e.g., a needle that is inserted into a port). The ultrasound machine 102 includes various components, some of which include the scanner 104, one or more processors 106, a display device 108, a memory 110, and a transceiver 112.

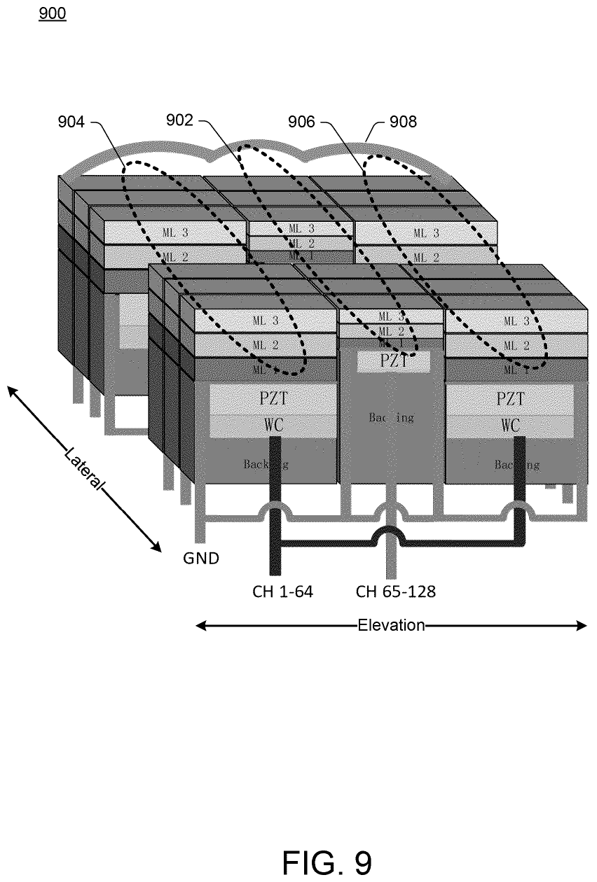

A user 114 (e.g., nurse, ultrasound technician, operator, sonographer, clinician, etc.) directs the scanner 104 toward a patient 116 to non-invasively scan internal bodily structures (e.g., patient anatomies such as organs, tissues, bones, etc.) of the patient 116, a port, an interventional instrument, etc., for testing, diagnostic, therapeutic, or procedural reasons, including determining port health. In some embodiments, the scanner 104 includes an ultrasound transducer array and electronics communicatively coupled to the ultrasound transducer array to transmit ultrasound signals to the patient's anatomy and receive ultrasound signals reflected from the patient's anatomy. In some embodiments, the scanner 104 is an ultrasound scanner, which can also be referred to as an ultrasound probe or transducer. In some embodiments, the scanner 104 is a multi-array scanner. For instance, a multi-array scanner in accordance with some embodiments can include one or more of the arrays described in U.S. patent application Ser. No. 18/613,694 filed on Mar. 22, 2024, entitled Multi-Dimensional and Multi-Frequency Ultrasound Transducers to Zhang et al., the disclosure of which is incorporated herein by reference in its entirety. A multi-array scanner in accordance with some embodiments can include one or more of the arrays described in U.S. patent application Ser. No. 17/561,313 filed on Dec. 23, 2021, entitled Array Architecture and Interconnection for Transducers to Li et al., the disclosure of which is incorporated herein by reference in its entirety. Further, multi-array scanners for determining port health with ultrasound are discussed below in more detail with respect to FIGS. 9-14.

The display device 108 is coupled to the processor 106, which can include any suitable processor, number of processors, or processor system, such as one or more central processing units (CPUs), graphics processing units (GPUs), vector processors, Reduced Instruction Set Computer (RISC) processors, Reduced Instruction Set Computer (CISC) processors, very long instruction word (VLIW) processors, etc. The processor 106 can execute instructions stored on memory 110 to perform operations disclosed herein for determining port health with ultrasound. For example, the processor 106 can process the reflected ultrasound signals to generate ultrasound data, including an ultrasound image. The display device 108 is configured to generate and display an ultrasound image (e.g., ultrasound image 118) of the anatomy and/or interventional instrument (e.g., a port or needle) based on the ultrasound data generated by the processor 106 from the reflected ultrasound signals detected by the scanner 104. In some embodiments, the ultrasound data includes the ultrasound image 118 or data representing the ultrasound image 118. The transceiver 112 can be configured to transmit, e.g., over a network maintained by a care facility, the ultrasound data and/or any data related to the ultrasound examination, such as medical worksheet data, a health status report of a port, etc., to a medical archiver (e.g., a vendor neutral archive (VNA)). In some embodiments, the transceiver 112 can receive data from the medical archiver, such as patient history data or previous examination data.

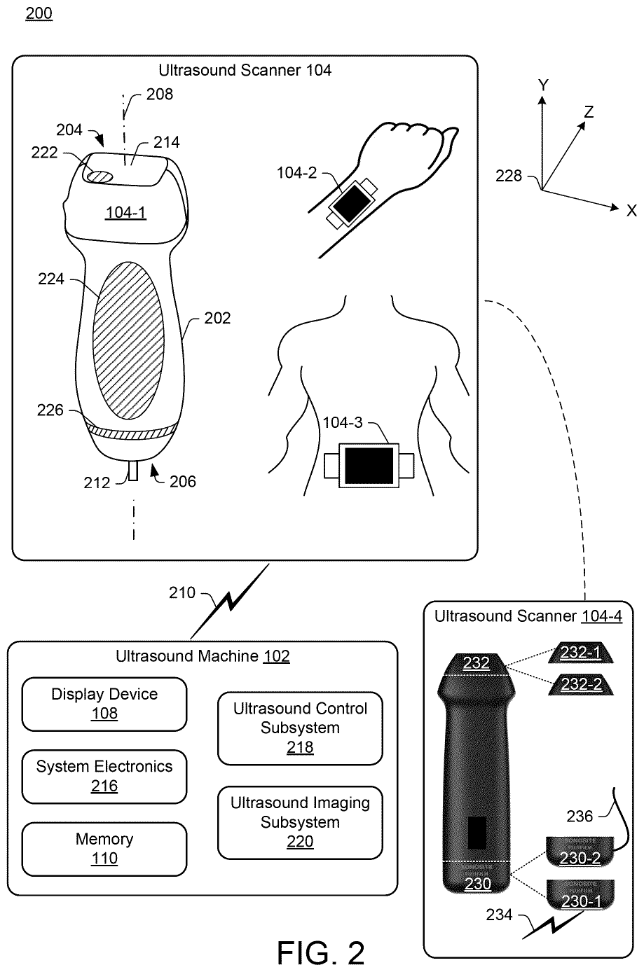

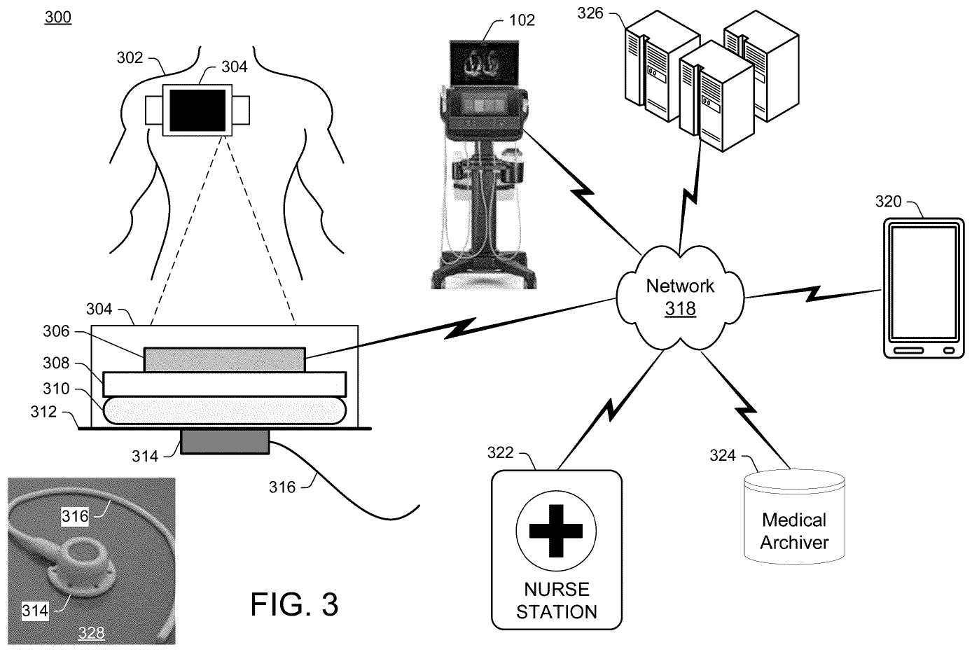

FIG. 2 illustrates an example implementation 200 of the ultrasound system illustrated in the environment 100 of FIG. 1. In the implementation 200, the scanner 104 (e.g., ultrasound scanner) can be any suitable type of ultrasound scanner. In some embodiments, the scanner 104 includes a scanner 104-1 configured for handheld operation, e.g., external to a patient's body. Other embodiments of the scanner 104, including scanner 104-2 and 104-3, include wearable ultrasound scanners (e.g., patch-based ultrasound scanners), that can be worn by a patient for testing, diagnostic, therapeutic, or procedural reasons, including long term monitoring for determining port health with ultrasound, and are discussed below in more detail with respect to FIGS. 3-6. Another example of the scanner 104 includes the ultrasound scanner 104-4, which is configured for handheld operation like the scanner 104-1 and includes removably attachable ultrasound arrays and removably attachable electronics for wired and/or wireless operation, as discussed below in more detail.

The scanner 104-1 includes an enclosure 202 extending between a distal end portion 204 and a proximal end portion 206. The enclosure 202 includes a central axis 208 (e.g., longitudinal axis) that intersects the distal end portion 204 and the proximal end portion 206. The central axis 208 corresponds to an axial direction of the scanner 104-1. The scanner 104-1 is electrically coupled to an ultrasound imaging system (e.g., the ultrasound machine 102) via a coupling 210. In some embodiments, the coupling 210 includes a cable that is attached to the proximal end portion 206 of the scanner 104-1 by a strain-relief element 212. In some embodiments, the coupling 210 includes a wireless coupling so that the scanner 104-1 is wirelessly coupled to the ultrasound imaging system and communicates with the ultrasound imaging system via one or more wireless transmitters, receivers, or transceivers over a wireless connection or network (e.g., Bluetooth™, Wi-Fi™, etc.).

A transducer assembly 214 having one or more transducer elements is electrically coupled to system electronics 216 in the ultrasound machine 102. In operation, the transducer assembly 214 transmits ultrasound energy from the one or more transducer elements toward a subject and receives ultrasound echoes from the subject. The ultrasound echoes are converted into electrical signals by the transducer element(s) and electrically transmitted to the system electronics 216 in the ultrasound machine 102 for processing and generation of one or more ultrasound images.

Capturing ultrasound data from a subject using a transducer assembly (e.g., the transducer assembly 214) generally includes generating ultrasound signals, transmitting ultrasound signals into the subject, and receiving ultrasound signals reflected by the subject. A wide range of frequencies of ultrasound can be used to capture ultrasound data, such as, for example, low-frequency ultrasound (e.g., less than 15 Megahertz (MHz)) and/or high-frequency ultrasound (e.g., greater than or equal to 15 MHz). A particular frequency range to use can readily be determined based on various factors, including, for example, depth of imaging, desired resolution, and so forth.

In some embodiments, the system electronics 216 include one or more processors (e.g., the processor(s) 106 from FIG. 1), integrated circuits, application-specific integrated circuits (ASICs), Field Programmable Gate Arrays (FPGAs), and power sources to support functioning of the ultrasound machine 102. In some embodiments, the ultrasound machine 102 also includes an ultrasound control subsystem 218 having one or more processors. At least one processor, FPGA, or ASIC can cause electrical signals to be transmitted to the transducer(s) of the scanner 104 to emit sound waves and also receives electrical pulses from the scanner 104 that were created from the returning echoes. One or more processors, FPGAs, or ASICs can process the raw data associated with the received electrical pulses and form an image that is sent to an ultrasound imaging subsystem 220, which causes the image (e.g., the image 118 in FIG. 1) to be displayed via the display device 108. Thus, the display device 108 displays ultrasound images from the ultrasound data processed by the processor(s) of the ultrasound control subsystem 218.

In some embodiments, the ultrasound machine 102 also includes one or more user input devices (e.g., a keyboard, a cursor control device, a microphone, a camera, touchscreen, etc.) that input data and enable taking measurements from the display device 108 of the ultrasound machine 102. The ultrasound machine 102 can also include a disk storage device (e.g., computer-readable storage media such as read-only memory (ROM), a Flash memory, a dynamic random-access memory (DRAM), a NOR memory, a static random-access memory (SRAM), a NAND memory, and so on) for storing the acquired ultrasound data. In some embodiments, the disk storage device includes the memory 110, which is local to the ultrasound machine 102. Alternatively, the memory 110 used for storing the acquisition data can be remote, such as on a remote server communicatively connected to the ultrasound machine 102. In addition, the ultrasound machine 102 can include a printer that prints the image from the displayed data. To avoid obscuring the techniques described herein, such user input devices, disk storage device, and printer are not shown in FIG. 2.

In some embodiments, the ultrasound scanner 104-1 in the implementation 200 also includes one or more pressure sensors 222 on the lens of the scanner 104-1, and one or more pressure sensors 224 on the enclosure 202 of the scanner 104-1. The pressure sensors 222 and 224 can include in, on, or under a sensor region any suitable type of sensors for determining a pressure. In one example, the pressure sensors 222 and 224 includes capacitive sensors that can measure a capacitance, or change in capacitance, caused by a user's touch or proximity of touch, as is common in touchscreen technologies. The pressure sensors 222 and 224 can generate sensor data indicative of a touch or pressure. The sensor data can include a binary indicator that indicates the presence and absence of a touch on the sensor. For instance, a “1” for sensor data can indicate that a pressure is sensed at the pressure sensor, and a “0” for the sensor data can indicate that a pressure is not sensed at the pressure sensor. Additionally or alternatively, the sensor data can include a multi-level indicator that indicates an amount of pressure on the sensor, such as an integer scale from zero to five. For instance, a “0” can indicate that no pressure is detected at the sensor, and a “1” can indicate a small amount of pressure is detected at the sensor. A “2” can indicate a larger amount of pressure is detected at the sensor than a “1”, and a “5” can indicate a maximum amount of pressure is detected at the sensor.

The pressure sensors 222 and 224 are illustrated in FIG. 2 as ellipses for clarity, and generally can be of any suitable shape and size and generate sensor data indicating pressure at any suitable number of points. For instance, in some embodiments, the pressure sensors 222 cover an exterior surface of the lens of the scanner 104-1 and can be used to determine when the scanner is placed against a patient. Additionally or alternatively, the pressure sensors 224 can substantially cover the enclosure 202 of the scanner 104-1 and can be used to determine when a clinician grabs the scanner 104-1 for use in an ultrasound examination (e.g., the clinician has a suitable grip on the scanner 104-1 to perform the ultrasound examination). The ultrasound system can use the sensor data from one or both of the pressure sensors 222 and 224 to generate a trigger signal that can be used for determining port health with ultrasound. For instance, when the sensor data from one or both of the pressure sensors 222 and 224 is above a threshold level, and/or the sensor data from the pressure sensors 224 indicate a grip pattern indicative of a human operating the scanner, the system can generate a trigger signal. The trigger signal can be used to cause the ultrasound system to enable one or more machine-learned models to generate a health status of a port, including a prediction of when the port will fail. For instance, the ultrasound system can, based on the trigger signal, determine from the ultrasound data one or more of an indication of infection of tissue proximate to the port, an amount of swelling of the tissue, an indication of congestion in the port, a measure of volume flow of the fluid or the additional fluid, a measure of the temperature of tissue proximate to the port, and a measure of pressure of the fluid or the additional fluid. One or more machine-learned models can make these determinations. Another machine-learned model can then process this data and generate the health status of the port, including a prediction of when the port will fail.

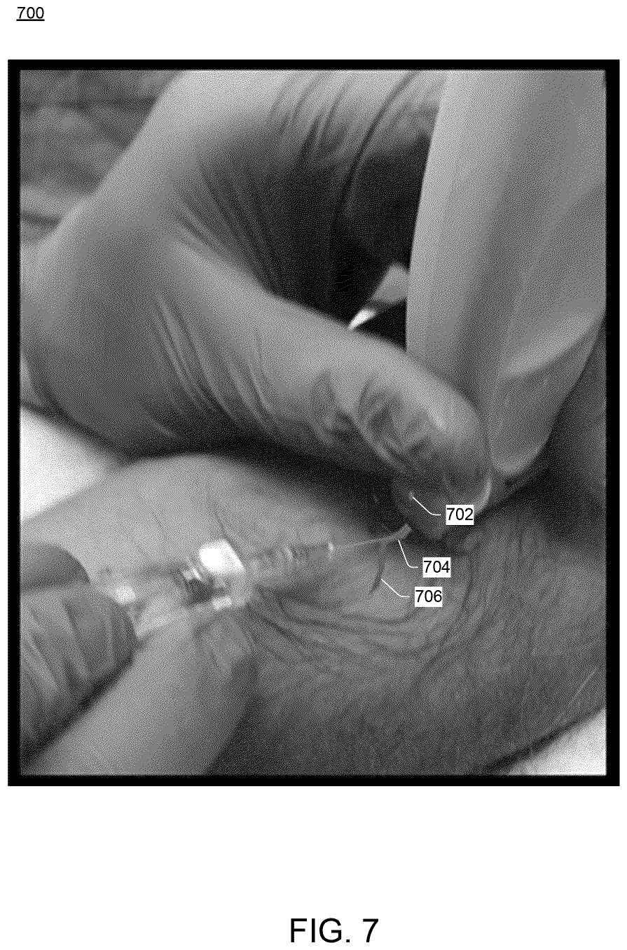

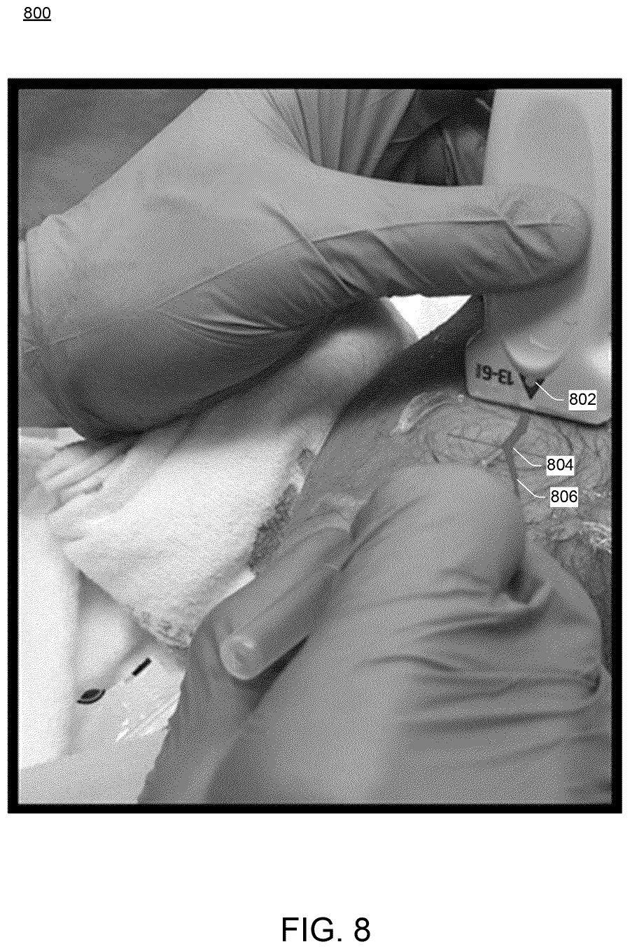

In some embodiments, the ultrasound system uses the trigger signal to enable one or more light sources (e.g., microelectromechanical systems (MEMS) lasers) for needle insertion guidance. For instance, the light sources can indicate a current position of the needle tip, under which array out of multiple arrays on the scanner the needle tip is positioned, indicate the position of a blood vessel, indicate an insertion point for the needle on the patient's skin, etc. The light sources can project light onto the patient's skin from the scanner, discussed below in more detail with respect to FIGS. 7 and 8.

In some embodiments, the scanner 104-1 includes an inertial measurement unit (IMU) 226 for generating positional data that determines a position and orientation of the scanner 104-1 in a coordinate system, e.g., the coordinate system 228 in FIG. 2. The IMU can include a combination of accelerometers, gyroscopes, and magnetometers, and generate positional data including data representing six degrees of freedom (6DOF), such as yaw, pitch, and roll angles in the coordinate system. Typically, 6DOF refers to the freedom of movement of a body in three-dimensional space. For example, the body is free to change position as forward/backward (surge), up/down (heave), left/right (sway) translation in three perpendicular axes, combined with changes in orientation through rotation about three perpendicular axes, often termed yaw (normal axis), pitch (transverse axis), and roll (longitudinal axis). Additionally or alternatively, the ultrasound system can include a camera and fiducial markers on the scanner 104-1 (not shown in FIG. 2) to determine the positional data for the ultrasound scanner 104-1. In one example, the system generates, based on the positional data, a trigger signal as described above. For some embodiments the positional data can indicate that the scanner 104 is within a threshold distance of the patient, and the trigger signal can be used by the ultrasound system to enable one or more machine-learned models, e.g., to generate a health status report for a port, as described above.

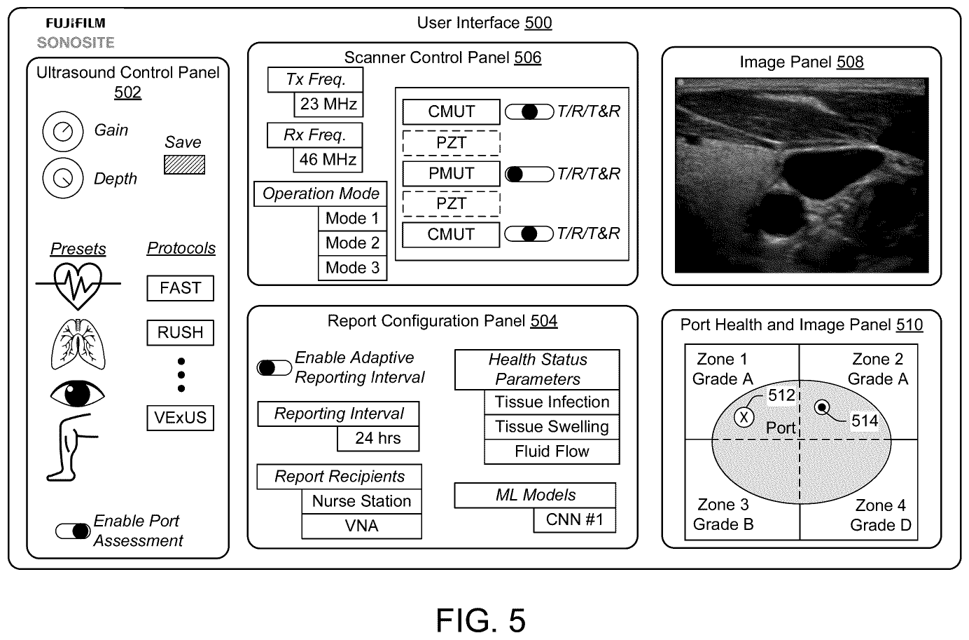

A trigger signal generated by the system, e.g., due to pressure data and/or positional data as described above, can be used to expedite the workflow of the system. In some embodiments, responsive to a trigger signal, the system automatically requests a patient history, such as from a medical archiver (e.g., a VNA). The patient history can include previously-generated port health status reports for the patient, which can be displayed by the system for comparison against a current port health status report. In some embodiments, responsive to a trigger signal, the system causes a port health and image panel (e.g., the port health and image panel 510 in FIG. 5) to be displayed by a computing device. In another example, the system can, responsive to a trigger signal, enable one or more arrays of a scanner (e.g., the scanner 104), according to an operation mode of the system. Example operations modes are described below with respect to Table 1.

The scanner 104 also includes example scanners 104-2 and 104-3 that are coupled to the ultrasound machine via the coupling 210, e.g., via a wireless connection. The scanners 104-2 and 104-3 are examples of wearable ultrasound scanners that can be patient worn, such as patches that can be placed over a port that has been installed in a patient. The scanners 104-2 and 104-3 can be used during an ultrasound examination, e.g., for testing, diagnostic, therapeutic, or procedural reasons. Additionally or alternatively, the scanners 104-2 and 104-3 can be placed on a patient for longer term monitoring, e.g., days, weeks, or months, to continuously (e.g., periodically) monitor the health of a port and generate a health status report. Hence, the scanners 104-2 and 104-3 can be used remotely by the patient, so that the patient can forego visits to a care facility to determine a health status of a port. In some embodiments, in the case of a patch with wearable ultrasound scanners, the patch (and/or port) can include one or more communication interfaces (electronics) to send data from the wearable ultrasound scanners 104-2 and 104-3 to a remote location (e.g., a network) to provide the health status of the port. The sending of such data can be automatic upon uses of the scanner or at certain times (e.g., predetermined times, regular intervals, etc.). The communication interfaces can also be used to receive data from another source (e.g., medical machine, medical vital sign capture device, medicine receptacle, etc.) which can be combined and sent with the data from scanners 104-2 and 104-3. The health status report can include a prediction of when the port will fail. Hence, a patient can get the port repaired or replaced prior to the port failure, so that the patient does not need to miss or delay a procedure that requires the port, such as chemotherapy treatment or blood transfusion.

In some embodiments, the time intervals (e.g., periodic intervals) that the system generates a health status report are generated adaptively by the system. For example, the time intervals can be based on a previously-determined health of the port from the scanners 104-2 and 104-3. For instance, if the system determines a health status of the port that includes severe tissue swelling, infection, etc., and/or an expected (e.g., predicted) time of port failure that is short (e.g., within a few days), the system can generate new health statuses for the port more frequently than if no swelling or infection is detected, or if the expected time to failure for the port is long (e.g., in two months). In some embodiments, the scanners 104-2 and 104-3 can generate the health status reports without intervention by the patient, and automatically and without user intervention send a health status of the port to another party, such as a nurse station, doctor, medical archiver, etc.

The scanner 104 also includes the example scanner 104-4, which is configured for handheld operation like the scanner 104-1 and includes removably attachable electronics 230 and removably attachable ultrasound arrays 232. The removably attachable electronics 230 can be removed from the body of the scanner 104-4 and re-attached. Any suitable attachment mechanism can be used for removal and attachment of the removably attachable electronics 230 and the removably attachable ultrasound arrays 232 from/to the scanner 104-4, such as rails, dovetails, clamps, fasteners, gaskets, O-rings, etc. In some embodiments, the removably attachable electronics 230 and the removably attachable ultrasound arrays 232 can be attached to the body of the scanner 104-4 and maintain an IPX7-rated seal.

Examples of the removably attachable electronics 230 include removably attachable electronics 230-1 and 230-2. The removably attachable electronics 230-1 include circuitry so that the scanner 104-4 can communicate with an ultrasound machine, e.g., the ultrasound machine 102, over a wireless communication link 234. The removably attachable electronics 230-2 include circuitry so that the scanner 104-4 can communicate with an ultrasound machine, e.g., the ultrasound machine 102, over a wired communication link 236, such as via one or more cables. Hence, the scanner 104-4 can be reconfigured for use with different ultrasound machines, including ones that support wired coupling to the scanner 104-4 and others that support wireless coupling to the scanner 104-4.

Examples of the removably attachable ultrasound arrays 232 include removably attachable arrays 232-1 and 232-2. The removably attachable arrays 232-1 and 232-2 can include multi-array transducer assemblies (e.g., as discussed below with respect to FIGS. 9-14). Accordingly, the removably attachable arrays 232-1 and 232-2 can include multi-array transducer assemblies that include any combination of piezoelectric micromachined ultrasonic transducer (PMUT) array elements, lead zirconate titanate (PZT) array elements, and capacitive micromachined ultrasonic transducer (CMUT) array elements. In some embodiments, the removably attachable array 232-1 includes at least one array with PZT array elements and the removably attachable array 232-2 includes at least one array with CMUT array elements. In another example, the removably attachable array 232-1 includes at least one array with PMUT array elements and the removably attachable array 232-2 includes at least one array with CMUT array elements. In still another example, at least one of the removably attachable arrays 232-1 and 232-2 includes a first array with array elements selected from the group consisting of PZT, PMUT, and CMUT array elements, and a second array with additional array elements selected from the group consisting of PZT, PMUT, and CMUT array elements. The elements of the first array can be of a different type than the elements of the second array (e.g., the first array can include PMUT elements and the second array can include CMUT elements). Alternatively, the elements of the first array can be of a same type as the elements of the second array (e.g., the first array and the second array can both include PMUT elements). Hence, the user may not need to carry an assortment of different scanners, but instead carry the scanner 104-4 with removably attachable ultrasound arrays 232 that can be used for different types of examinations.

FIG. 3 illustrates some embodiments of a system in an environment 300 for determining port health with ultrasound in accordance with the present invention. The environment 300 includes a patient 302 who is wearing a wearable ultrasound scanner 304. The wearable ultrasound scanner 304 is an example of the scanners 104-2 and 104-3 in FIG. 2. The wearable ultrasound scanner 304 can be placed over a port installed on the patient 302 (e.g., the port having reservoir 314 and line 316, and the port illustrated at inlay 328).

The wearable ultrasound scanner 304 includes circuitry 306, as is illustrated in profile in FIG. 3. The circuitry 306 can include any suitable electronics to process and/or display ultrasound data generated by the wearable ultrasound scanner 304, and communicate data to another device, such as the ultrasound machine 102 or any of the computing devices illustrated in FIG. 3. For example, the circuitry 306 can include the system electronics 216, the memory 110, the ultrasound control subsystem 218, the ultrasound imaging subsystem 220, the processors 106, the transceiver 112, the display device 108, and the like. The circuitry 306 can communicate over the network 318, e.g., via Bluetooth™, Wi-Fi™, etc. In some embodiments, the network 318 includes a network maintained by a care facility, e.g., an intranet. Additionally or alternatively, the network 318 can include the Internet.

The wearable ultrasound scanner 304 also includes one or more transducer arrays 308. The transducer arrays 308 can include any suitable type of transducer array, including one or more transducer arrays that include one or more elements selected from the group consisting of piezoelectric micromachined ultrasonic transducer (PMUT) array elements, lead zirconate titanate (PZT) array elements, and capacitive micromachined ultrasonic transducer (CMUT) array elements. In an example, the wearable ultrasound scanner 304 is a multi-array ultrasound scanner that includes one or more transducer arrays having PZT array elements and one or more additional transducer arrays having PMUT array elements. In another example, the wearable ultrasound scanner 304 is a multi-array ultrasound scanner that includes one or more transducer arrays having PZT array elements and one or more additional transducer arrays having CMUT array elements. In still another example, the wearable ultrasound scanner 304 is a multi-array ultrasound scanner that includes one or more transducer arrays having PMUT array elements and one or more additional transducer arrays having CMUT array elements.

In some embodiments, the wearable ultrasound scanner 304 is a multi-array ultrasound scanner that includes multiple transducer arrays having PZT array elements. In some embodiments, the wearable ultrasound scanner 304 is a multi-array ultrasound scanner that includes multiple transducer arrays having PMUT array elements. In some embodiments, the wearable ultrasound scanner 304 is a multi-array ultrasound scanner that includes multiple transducer arrays having CMUT array elements.

The wearable ultrasound scanner 304 can include a lens (not shown in FIG. 3 for clarity). The lens can be placed over the transducer arrays 308 and facing the patient. In some embodiments, the wearable ultrasound scanner 304 also includes a coupling agent 310 that couples ultrasound from the transducer arrays 308 to the patient anatomy and the port having reservoir 314 and line 316, as well as reflections of the ultrasound from the patient anatomy and the port back to the transducer arrays 308. The coupling agent 310 can be encapsulated in a packet (e.g., a gel pack). In some embodiments, the gel pack is affixed to a tegaderm pad or patch. In some embodiments, when determining port health with ultrasound, the packet can be affixed to the transducer arrays 308 or a lens covering the transducer arrays. The packet can be held in place with pressure, a pocket or recess to hold the packet, a clip or stand to retain the packet, or combinations thereof. In some embodiments, the coupling agent 310 can lack a bounding container, like a packet, and instead include a gel or paste.

The wearable ultrasound scanner 304 is placed against the patient skin 312, over the port having reservoir 314 and line 316. A photograph of an example of the port is illustrated in the inlay 328 in FIG. 3. The port's reservoir 314 can include one or more access points for a needle, and the port's line 316 can be inserted into a blood vessel of the patient 302. Hence, fluid can be drawn from the blood vessel via the port, and/or inserted into the blood vessel via the port. The wearable ultrasound scanner 304 can be affixed to the patient skin 312 via any suitable mechanism, including glue, tape, a tegaderm patch, a bandage wrapped around the patient, etc.

The wearable ultrasound scanner 304 can generate ultrasound and receive reflections of the ultrasound from the patient's 302 anatomy and the port's reservoir 314 and line 316. Based on the ultrasound reflections, the wearable ultrasound scanner 304 can determine a health status of the port. For example, the wearable ultrasound scanner 304 can determine, based on the ultrasound data, one or more parameters, including an indication of infection of tissue proximate to the port, an amount of swelling of the tissue, an indication of congestion in the port, a measure of volume flow of the fluid or the additional fluid, a measure of the temperature of tissue proximate to the port, and a measure of pressure of the fluid or the additional fluid. In some embodiments, the circuitry 306 implements one or more machine-learned models to determine these parameters. The circuitry 306 can also implement a machine-learned model (e.g., a convolutional neural network) to generate, based on one or more of these parameters, a health status report for the port. The health status report can include an estimated (e.g., predicted) time to failure for the port. Additionally or alternatively, the health status report can include a grade or score for the health of the port, such as a letter grade in the scale of “A” to “F”, or a number score in the scale of one to ten. Additionally or alternatively, the health status report can include an indication that a port has already failed and should not be used for fluid insertion or removal. In some embodiments, the wearable ultrasound scanner 304 can generate a recommendation, such as a recommended insertion point of the needle, a recommended date for a follow-up examination, etc.

The wearable ultrasound scanner 304 can communicate any suitable data, such as the health status report of a port, over the network 318 to a computing device coupled to the network 318, including the computing device 320 (e.g., a smart phone or tablet, such as a doctor's personal computing device), a device at a nurse station 322, a medical archiver 324, a server 326, and the ultrasound machine 102. By communicating the health status report to the nurse station 322, care facility staff can be kept up to date on the health of the patient's port and make appropriate scheduling decisions. For instance, the staff can adjust the timing of a scheduled treatment so that it is completed before the health of the port deteriorates below a threshold level or reschedule a scheduled treatment until after the port has been repaired or replaced.

The server 326 can include one or more server devices, such as a server system maintained by a care facility. A clinician may have access to the server 326 to review a health status report for a patient. The medical archiver 324 can maintain patient data and update the patient's 302 records with a health status report of a port provided by the wearable ultrasound scanner 304. The medical archiver 324 can also provide previous patient data for comparison to current patient data. For instance, a clinician or the patient 302 can compare a current health status report generated with the wearable ultrasound scanner 304 to previous health status reports generated with the wearable ultrasound scanner 304 or another ultrasound scanner.

In some embodiments, a computing device (e.g., the computing device 320) can display guidance to a user to help place the wearable ultrasound scanner 304 at a location and orientation on the patient 302 to image the port having the reservoir 314 and the line 316 to generate a health status report of the port. For instance, the computing device 320 can display directional arrows, angles, etc. to align the wearable ultrasound scanner 304 with the port so that a desired view is achieved in an ultrasound image. In some embodiments, the computing device 320 displays an ultrasound image having a view that the user can match by maneuvering the wearable ultrasound scanner 304 for placement on the patient 302. The wearable ultrasound scanner 304 can include any suitable glue, tape, binder, etc. to anchor the wearable ultrasound scanner 304 to the patient 302.

In some embodiments, the wearable ultrasound scanner 304 can be used for other applications than determining port health with ultrasound. For example, in some embodiments, the wearable ultrasound scanner 304 is used for monitoring fluid status in a patient. In another example, in some other embodiments, the wearable ultrasound scanner 304 can be used for cardiac monitoring, such as to monitor the size of a patient's heart, or a left ventricle parameter, such as ejection fraction. In another example, in yet some other embodiments, the wearable ultrasound scanner 304 can be used for respiratory monitoring, such as for determining lung sliding or b-line assessment. Monitoring can be done during a procedure, after a procedure, or before a procedure, including for diagnostic or therapeutic reasons, such as for pain management, neuropathy treatment, or to break up calcium deposits. In some embodiments, the wearable ultrasound scanner 304 is applied to a patient's temporal region for monitoring intercranial pressure and/or optic nerve assessment. In embodiments, the system includes a vest that includes multiple wearable ultrasound scanners, such as multiple wearable ultrasound scanners 403. The vest can be worn by a patient and used for monitoring abdominal, chest, cardiac, and other anatomies for diagnostic, therapeutic, or procedural reasons.

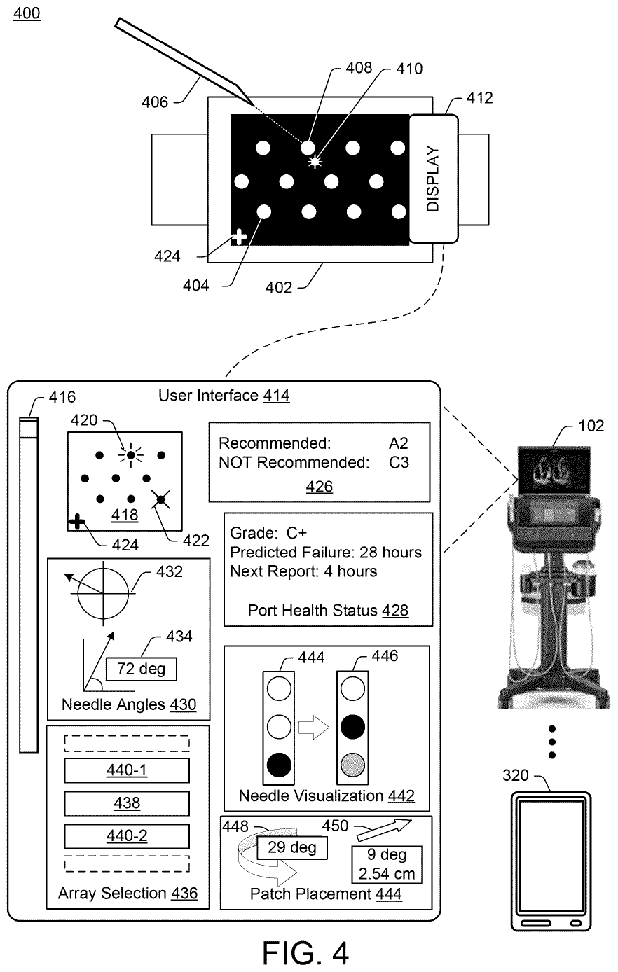

FIG. 4 illustrates some embodiments of a system 400 for determining port health with ultrasound. The system 400 includes a wearable ultrasound scanner 402, which is an example of the wearable ultrasound scanner 304 in FIG. 3 and the wearable scanners 104-2 and 104-3 in FIG. 2. In some embodiments, the wearable ultrasound scanner 402 includes a patch that can be affixed to a patient over a port installed in the patient. In some embodiments, the wearable ultrasound scanner 402 includes multiple access holes 404 through which an interventional instrument 406, such as a needle, can be inserted into a port to supply fluid into the patient and/or remove fluid from the patient. For clarity, only one of the holes is designated as 404.

In some embodiments, the system 400 generates a recommendation that includes a recommended hole 408 for insertion of the interventional instrument 406 and indicates the recommended hole 408 by illuminating one or more light sources 410 that are proximate to the recommended hole 408. In some embodiments, the system 400 implements one or more machine-learned models to generate the recommendation that includes the recommended hole 408. For instance, the machine-learned model can include a convolutional neural network that can process one or more parameters determined based on ultrasound generated by the wearable ultrasound scanner 402. Example parameters include an indication of infection of tissue proximate to the port, an amount of swelling of the tissue, an indication of congestion in the port, a measure of volume flow of the fluid or the additional fluid, a measure of the temperature of tissue proximate to the port, and a measure of pressure of fluid input to, or removed from, the patient. One or more of these parameters can be concatenated into an input vector that is processed by the convolutional neural network. Alternatively, some of these parameters can be concatenated into an input vector that is processed by layers of the convolutional neural network to generate a feature vector. The others of these parameters can be provided as conditional inputs to the convolutional neural network and concatenated with the feature vector that the convolutional neural network generates at the output of said layers of the convolutional neural network. The resulting concatenated vector can be processed by additional layers of the convolutional neural network and used to generate the recommended hole 408. In this way, the convolutional neural network can process some of the parameters at the top layer, and others of the parameters at intermediate layers.

Additionally or alternatively, the system 400 can implement one or more machine-learned models to generate a health status report of the port. In some embodiments, the system can then generate, based on the health status report, the recommended hole 408 for insertion of the interventional instrument 406, e.g., a needle. By recommending one of the holes for insertion of the needle, the system can prevent overuse of an area of the port and prolong the port's time to failure. By prolonging the time to failure for the port, the patient may be able to forego the installation or repair of the port, thus reducing or eliminating the need to reschedule or delay procedures that require port access. Further, the system can instruct the user to avoid areas of the port that are proximate to infected or inflamed tissue, thus reducing pain and discomfort for the patient. In some embodiments, the system can determine the recommended hole 408 for insertion so as to avoid these areas. Additionally or alternatively, the system can determine the recommended hole 408 for insertion so as to avoid a location on the port that was used for one or more previous needle insertions.

Note that the recommendations from monitoring with ultrasound are not limited to provided recommended holes for insertion of a needle or other instrument. For example, the recommendations can include a recommendation as to the substance to inject into a port. Such a substance can be used to address inflammation in the tissue around the port. As another example, the recommendations can include whether to attach a certain type of pump to the port (e.g., pain pump, infusion pump, chemotherapy pump, etc.).

The wearable ultrasound scanner 402 can also include a display 412. In some embodiments, the display 412 is removably attached to the wearable ultrasound scanner 402. Hence, the wearable ultrasound scanner 402 can be disposable, and the display device can be sterilized for reuse with another wearable ultrasound scanner. In some embodiments, the display 412 can be removably attached to the wearable ultrasound scanner 402 via any suitable mechanism, such as hook-and-loop fasteners, molded plastic parts that clasp the display 412, magnets, etc.

The display 412 can display a user interface 414. Additionally or alternatively, the user interface 414 can be displayed by another device, such as the ultrasound machine 102 and/or the computing device 320. In some embodiments, the user interface 414 includes a slider 416 to enable a user to scroll through content displayed on the user interface 414. In some embodiments, the user interface 414 includes a visual representation 418 that represents the holes 404 of the wearable ultrasound scanner 402. In the example in FIG. 4, the visual representation 418 includes a pattern representing the holes 404. The recommended hole 408 for insertion is indicated by a light source 420 on the visual representation 418. The light source 420 on the user interface 414 can indicate the recommended hole 408 for insertion in a similar manner as the light source 410 on the wearable ultrasound scanner 402, such as by blinking on and off, blinking in a pattern of on and off, changing color, changing intensity, etc.

In some embodiments, the visual representation 418 also includes a recommendation 422 that indicates one of the insertion holes to avoid (e.g., not to use) for needle insertion. The system 400 can determine the recommendation 422 for an insertion hole to avoid based on a history of needle insertions. For example, if one of the holes 404 was used for needle insertion in a previous treatment, then the system can determine not to use that hole for a needle insertion for a current treatment. Thus, the system can prevent the overuse of an insertion hole to prolong the life of the port and reduce the impact on the patient's tissue. In some embodiments, the visual representation 418 displays a registration mark 424 to align the orientation of the holes of the visual representation 418 with the holes of the wearable ultrasound scanner 402, which can also include the registration mark 424 (or a similar, matching registration mark).

In some embodiments, the user interface 414 also includes a panel 426 that can indicate recommended holes for insertion (e.g., the hole indicated by the light source 420) and holes not recommended for insertion (e.g., the hole indicated by the recommendation 422). In the example in FIG. 4, the panel 426 displays text that indicates the holes to use and not to use. The text indicates a row with a letter and a column with a number. For instance, “A2” refers to the hole 408 and “C3” refers to the hole indicated by the recommendation 422.

In some embodiments, the user interface 414 also includes a port health status panel 428 that can include any data generated by the system 400 as part of a health status report for a port. For instance, the port health status panel 428 in FIG. 4 includes a grade of C+ for the port (e.g., on a scale of A-F), and a predicted time to failure for the port of 28 hours. The port health status panel 428 also includes an indication that the system 400 will generate a next health status report for the port in 4 hours from the present time.

In some embodiments, the user interface 414 also includes a needle angles panel 430 that can include any suitable data to indicate a recommended orientation for a needle that is inserted into the port, e.g., by inserting the needle into the recommended hole 408. In the example in FIG. 4, the needle angles panel 430 includes a visual representation 432 that indicates a recommended angle of the needle (e.g., the interventional instrument 406) in the horizontal plane (e.g., coplanar with the wearable ultrasound scanner 402). The angle can be relative to a coordinate system, such as axes that represent edges of the wearable ultrasound scanner 402, or the X-Y axes of the coordinate system 228 in FIG. 2. The needle angles panel 430 includes a visual representation 434 that indicates a recommended angle of the needle (e.g., the interventional instrument 406) in a vertical plane, e.g., a steepness angle. The angle can be in a Z-dimension of the coordinate system 228 in FIG. 2. In FIG. 4, the visual representation 434 includes both a graphic of the angle and text indicating that the recommended angle is 72 degrees.

In some embodiments, the user interface 414 also includes an array selection panel 436 that can include any suitable input or control to configure one or more arrays of the wearable ultrasound scanner 402. The array selection panel 436 in FIG. 4 includes a visual representation of five arrays of a multi-array transducer included in the wearable ultrasound scanner 402. A user can enable one or more of the five arrays by tapping on the visual representation for an array. For instance, the center array 438 is indicated as enabled, and two arrays 440-1 and 440-2 immediately adjacent to the center array 438 are also indicated as enabled (evidenced by the solid boxes for these arrays). The two outer-most arrays are indicated as not enabled, evidenced by the dashed boxes for these arrays.

In some embodiments, the user interface 414 also includes a needle visualization panel 442 that can include any suitable visual aids to visualize the needle 406, including the tip of the needle 406. In the example in FIG. 4, the needle visualization panel 442 includes a first visual representation 444 and a second visual representation 446 useful for out-of-plane needle visualization. The visual representations 444 and 446 each include three circles. The fill content of the circles indicates if the needle tip has been detected by the arrays, 438, 440-1, and 440-2. For instance, the top circle of the visual representations 444 and 446 corresponds to the array 440-1, the middle circle of the visual representations 444 and 446 corresponds to the array 438, and the bottom circle of the visual representations 444 and 446 corresponds to the array 440-2. Black fill content of a circle indicates that the tip of the needle 406 is currently detected by the array corresponding to the circle. Grey fill content of a circle indicates that the tip of the needle 406 was previously detected by the array corresponding to the circle and that the shaft of the needle 406 is currently detected by the circle. White fill content of a circle indicates that the needle 406 has not been detected by the array corresponding to the circle. In the example in FIG. 4, during operation the needle visualization panel 442 can first display the visual representation 444 to indicate that the tip of the needle 406 is detected by the array 440-2. When the tip of the needle passes through the imaging plane of the array 440-2, and into the imaging plane of the array 338, the needle visualization panel 442 can then display the visual representation 446. Accordingly, the needle visualization panel 442 can provide visual assistance to indicate a current position of the tip of the needle 406, as well as a trajectory of the needle 406.

In some embodiments, the user interface 414 also includes a patch placement panel 444 that can include any suitable visual aids to guide a user to place a wearable ultrasound scanner (e.g., the wearable ultrasound scanner 402) on a patient. In the example in FIG. 4, the patch placement panel 444 includes a first visual representation 448 that indicates to rotate the patch counter-clockwise by 29 degrees, and a second visual representation 450 that indicates to translate (e.g., move) the patch by 2.54 cm in a direction corresponding to a 9 degree vector. The system can generate the visual aids and guidance in any suitable way. In some embodiments, the system implements one or more machine-learned models that process ultrasound images generated by the wearable ultrasound scanner 402 as it is being placed on the patient. The machine-learned model can generate the guidance instructions as images, text, icons, combinations thereof, and the like. In some embodiments, the system uses registration marks on the patient, such as temporary tattoos or fiducial markers, to generate the guidance instructions. For example, the system can include one or more cameras that can image the registration marks on the patient and the registration mark 424 on the wearable ultrasound scanner 402. A machine-learned model can then generate the guidance instructions by processing the images from the cameras. Additionally or alternatively, the machine-learned model can process ultrasound images generated by the wearable ultrasound scanner 402 during its placement to generate the guidance instructions. For instance, a first image can be provided as an input to a top layer of a convolutional neural network (CNN) and a second image can be provided as a secondary input to a subsequent layer of the CNN and concatenated with a feature vector generated at the output of the subsequent layer based on the first image. Based on these images input to the CNN, it can generate the guidance instructions.

FIG. 5 illustrates some embodiments of a user interface 500 of a system for determining port health with ultrasound. The user interface can be displayed on any suitable computing device of the system for determining port health with ultrasound, including one or more of the ultrasound machine 102, the computing device 320, the server 326, and the wearable ultrasound device 402. In some embodiments, the interface 500 includes an ultrasound control panel 502, a report configuration panel 504, a scanner control panel 506, an image panel 508, and a port health and image panel 510.

In some embodiments, the ultrasound control panel 502 can include any suitable controls for configuring an ultrasound system for determining port health with ultrasound. In the example in FIG. 5, the ultrasound control panel 502 includes ultrasound controls for adjusting gain and depth, saving an image, and selecting examination presets. The examination presets are represented by selectable icons for a cardiac examination, a respiratory examination, an ocular examination, and a muscular-skeletal examination. These examination presets, when selected, can configure the ultrasound machine with predetermined values of gain and depth, and other imaging parameters (e.g., beamformer settings, filter coefficients, amplitude settings, etc.). The ultrasound control panel 502 also includes controls for selecting ultrasound protocols, including a Focused Assessment with Sonography for Trauma (FAST) protocol, a Rapid Ultrasound for Shock and Hypotension (RUSH) protocol, and a Venous Congestion Evaluation using Ultrasound (VExUS) protocol. A user can select one of these example protocols, and in response, the system can configure itself for an examination in accordance with the protocol, including to display a protocol panel in the user interface 500 (not shown for clarity) with guided steps needed to complete the selected protocol. The ultrasound control panel 502 also includes a selection (e.g., an electronic rocker switch) to enable port assessment.

Responsive to the selection to enable port assessment (e.g., via the rocker switch in the ultrasound control panel 502), the user interface 500 displays the report configuration panel 504. The report configuration panel 504 can display any suitable option, control, or setting to configure determining port health with ultrasound. In the example in FIG. 5, the report configuration panel 504 includes an electronic rocker switch to enable an adaptive reporting interval. When selected, the system can be adaptive when determining a time (e.g., time period) to generate and communicate a health status report of a port adaptively, e.g., based on the health status of the port. For instance, if the system determines a port is likely to fail within the next three days, the system can increase the rate at which the system generates and communicates a health status of the port, compared to if the system determines the port is estimated to fail in three months. As indicated in the example in FIG. 5, the adaptive reporting interval is disabled, and the reporting interval is set via a drop-down menu to 24 hours. Hence, in this configuration, the system can generate, based on ultrasound from a wearable ultrasound scanner, a health status of a port covered by the wearable ultrasound scanner every 24 hours, and communicate the health status of the port to a computing device, such as an ultrasound machine, clinician's personal computing device, nursing station, medical archiver, and the like. In the example in FIG. 5, a user has configured the system to send the report to a nurse's station and a vendor neutral archive (VNA), as evidenced by the drop-down menu selections in the report configuration panel 504.

The report configuration panel 504 also includes options for selection of the parameters used by the system to generate the health status of the port. In the example in FIG. 5, the system is configured to generate the health status report of the port based on tissue infection, tissue swelling, and fluid flow, and use a machine-learned model named CNN #1, as indicated by the drop-down menu selections in the report configuration panel 504. In this configuration, the system can enable the machine-learned model CNN #1 to process one or more ultrasound images generated with a wearable ultrasound scanner and generate the health status report by determining from the images measures of tissue infection and tissue swelling for tissue proximate to the port, and fluid flow for fluid in the port. In some embodiments, the report configuration panel 504 includes an option for automatically determining the health status parameters used for determining the health status of the port, and selecting the machine-learned model that generates the health status report (not shown in FIG. 5 for clarity).

In some embodiments, the user interface 500 also includes the scanner control panel 506 that includes controls and selections for configuring one or more arrays of a transducer assembly of an ultrasound scanner, including a wearable ultrasound scanner and a handheld ultrasound scanner. In some embodiments, the scanner control panel 506 includes options to select transmit and receive frequencies for one or more arrays of an ultrasound scanner. In the example in FIG. 5, a transmit frequency is set via a drop-down menu to 23 MHz, and a receive frequency is set via a drop-down menu to 46 MHz. In some embodiments, the scanner control panel 506 also includes options for enabling and configuring one or more arrays of a multi-array transducer. In the example in FIG. 5, the multi-array scanner includes five arrays (or sub-arrays), including a center array comprised of PMUT array elements, two adjacent arrays comprised of PZT array elements, and two outer arrays comprised of CMUT array elements. As indicated by the dashed lines, the PZT arrays are disabled, and as indicated by the solid lines, the PMUT and CMUT arrays are enabled. In some embodiments, a user can enable and disable an array by touching or tapping on the visual representation for the array. The scanner control panel 506 also includes options (e.g., three-position electronic rocker switches) to configure the enabled arrays for transmission, reception, or both transmission and reception. In the example in FIG. 5, the PMUT array (e.g., the center array) is configured to transmit, and the CMUT arrays (e.g., the outer arrays) are configured to receive. In some embodiments, the PMUT arrays have better transmit sensitivity (in terms of power efficiency) than the CMUT arrays, while the CMUT arrays have better receive sensitivity (in terms of signal strength) than the PMUT arrays.

In some embodiments, the scanner control panel 506 also includes a drop-down menu to enable the ultrasound scanner according to an operation mode. Example operation modes are described below with respect to Table 1, and include Mode 1 which can enable at least one array of an ultrasound scanner as a linear array and at least one additional array of the ultrasound scanner as a phased array, Mode 2 which can enable the arrays for broadband tissue harmonic imaging (broadband THI), and Mode 3 which can enable the arrays for full-aperture broadband THI operation.

In some embodiments, the user interface 500 also includes the image panel 508 for displaying any suitable type and number of ultrasound images. In the example in FIG. 5, the image panel 508 displays a B-mode image that includes blood vessels. A port can be inserted into the patient, and one end of the port can be inserted into a blood vessel of the image to insert and/or draw fluid.

In some embodiments, the user interface 500 also includes the port health and image panel 510 for indicating a health status of a port. The port health and image panel 510 includes a visual representation of a port, in this case an ellipse. The visual representation can include any suitable type of visual representation, such as an illustration, a photograph, an animation, an ultrasound image, etc. The port and surrounding tissue of the port are broken up into four zones 1-4, and the system assigns each of the four zones 1-4 a grade as part of generating a health status report of a port. Zones 1 and 2 are assigned an A grade, zone 3 is assigned a B grade, and zone 4 is assigned a D grade. Further, as an example, the system illustrates the previous needle insertion point 512 in zone 1, e.g., from a previous procedure, treatment, or examination. To avoid overuse of the port and tissue near the previous needle insertion point 512 in zone 1, and based on the health status of the port generated by the system (e.g., an A grade in zone 2), in some embodiments, the system recommends an insertion point 514 for the port in zone 2. In some embodiments, a wearable ultrasound scanner includes insertion holes (e.g., the access holes 404 in FIG. 4), and the system recommends one of the access holes for insertion of a needle, including guidance for needle orientation, so that the needle tip hits the insertion point 514.

In some embodiments, the user interface 500 includes a panel or other area that shows one or more patches that are available for monitoring. For example, the user interface 500 can show patches 1-5. In some embodiments, the user is able to select one (or more) of the patches on the user interface 500, which then causes data or other feedback from the selected patch(es) to be displayed in the user interface 500.

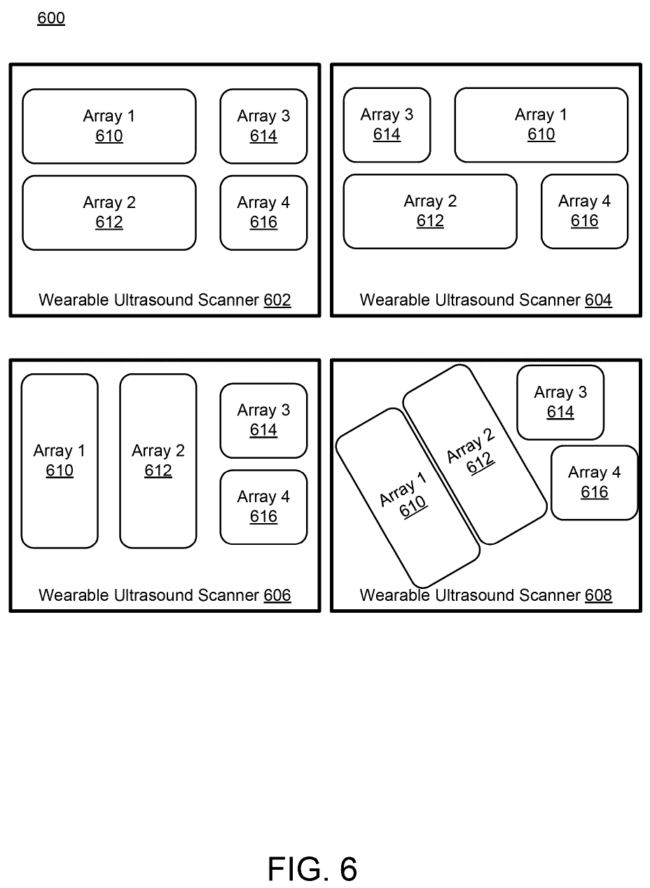

FIG. 6 illustrates some embodiments of configurations 600 of a reconfigurable wearable ultrasound scanner for determining port health with ultrasound. The configurations 600 include reconfigurable wearable ultrasound scanners 602-608, each of which include transducer arrays 610-616. In some embodiments, the transducer arrays 610-616 can include any suitable number of arrays. In some embodiments, one or more of the transducer arrays 610-616 include a multi-array transducer. A multi-array transducer can include any combination of PZT, PMUT, and CMUT arrays, such as is described below with respect to FIGS. 9-14. The transducer arrays 610-616 can be removably attached to a wearable ultrasound scanner, such as a patch-based wearable ultrasound scanner as previously described (e.g., the wearable ultrasound scanners 104-2, 104-2, 304, and 402). For example, the transducer arrays 610-616 can be removed from a wearable ultrasound scanner, repositioned or reoriented, and again attached to the wearable ultrasound scanner for use. The reconfigurable wearable ultrasound scanners 602-608 in FIG. 6 illustrate four examples in which the transducer arrays 610-616 are arranged in different orientations. The transducer arrays 610-616 can be removably attached to a wearable ultrasound scanner via any suitable mechanism, such as hook-and-loop fasteners, molded plastic parts that clasp the transducer arrays 610-616, magnets, etc.

By reconfiguring one or more of the transducer arrays 610-616 on the wearable ultrasound scanner, the wearable ultrasound scanner can remain on the patient and the arrays can be rotated and/or translated on the wearable ultrasound scanner. Hence, the wearable ultrasound scanner does not need to be removed from the patient to configure the system to obtain ultrasound images from different perspectives, or images of different anatomies, or images with different types of arrays. Further, one or more of the transducer arrays 610-616 can be removed from the wearable ultrasound scanner when it is not needed. For instance, suppose a certain procedure uses a CMUT transducer array and the procedure will not be performed for three months. During the three months, one or more PMUT transducer arrays can be attached to the wearable ultrasound scanner and used for port monitoring. The CMUT transducer array can then be attached to the wearable ultrasound scanner at the expiration of the three months to perform the procedure. In some embodiments, one or more of the transducer arrays 610-616 can be removed and replaced based on the type of treatment. For instance, a first array can be selected and installed for diagnostic purposes on the wearable ultrasound scanner. The first array can later be removed and replaced with a second array for therapeutic purposes. Hence, the transducer arrays 610-616 can be removably attached and replaced on the wearable ultrasound scanner to track the patient's treatment progress, without requiring removal of wearable ultrasound scanner from the patient.

In some embodiments, the transducer arrays can be positioned in a particular orientation. For example, the transducer arrays can be positioned to affect imaging that is to be performed, such as, biplane imaging. Biplane imaging can be useful when inserting a needle by providing an indication that the needle is in plane.

In some embodiments, patches having one or more transducer arrays can be combined. Such a combination can results in the same configurations of transducer arrays in FIG. 6 or other configurations. In some embodiments, the patches are combined by interleaving ultrasound data generated by different patches and processing the ultrasound data by a processing system in a joint manner, e.g., by treating the different patches as nodes in a synthetic aperture sensor system.

FIG. 7 illustrates an environment 700 with an example ultrasound scanner used for in-plane needle insertion. Referring to FIG. 7, the ultrasound scanner includes a light source 702. An example of the light source 702 includes a microelectromechanical systems (MEMS) emitter (e.g., a MEMS laser). The light source 702 projects a light onto the patient skin to indicate an insertion point 704 and trace a blood vessel 706. The ultrasound scanner can include a multi-array transducer, as is described below with respect to FIGS. 9-14. In some embodiments, the light source 702 can indicate a current position of the tip of the needle based on which array of the multi-array ultrasound scanner detects the needle tip in its imaging plane. For instance, the multi-array ultrasound scanner can include three arrays: a left array, a center array, and a right array. When the needle is in the imaging plane of the center array, the light source 702 can emit a green light. However, if the needle moves to the imaging plane of the left array or the right array, the light source 702 can change the color of the light emitted to red. In some embodiments, the color of the light emitted by the light source 702 corresponds to the array in which the needle is in plane. For instance, green can indicate that the needle is in the imaging plane of the center array, red can indicate that the needle is in the imaging plane of the left array, and orange can indicate that the needle is in the imaging plane of the right array. Additionally or alternatively, the light source 702 can change its intensity and/or blinking pattern to indicate which array's imaging plane corresponds to the current needle tip position. In this way, the light source 702 can guide the user to maintain the needle in-plane.

In some embodiments, the light source 702 includes multiple light sources spatially separated on the ultrasound scanner, such as side-by-side (not shown in FIG. 7 for clarity). Each of the arrays can correspond to one of the light sources. When the needle is in the imaging plane of one of the arrays, the system can cause the light source that corresponds to that array to project light. Hence, the user can determine if they are inserting the needle straight with respect to the lateral dimension of the ultrasound scanner by inspection of which light source is projecting light, and/or the color of the light, and/or the blinking pattern of the light.

FIG. 8 illustrates an environment 800 with an example ultrasound scanner used for out-of-plane needle insertion. The ultrasound scanner includes a light source 802. An example of the light source 802 includes a MEMS emitter (e.g., a MEMS laser). The light source 802 projects a light onto the patient skin to indicate an insertion point 804 and trace a blood vessel 806. The ultrasound scanner can include a multi-array transducer, as is described below with respect to FIGS. 9-14. In some embodiments, the light source 802 can indicate a current position of the tip of the needle based on which array of the multi-array ultrasound scanner detects the needle tip crossing its imaging plane. For instance, the multi-array ultrasound scanner can include three arrays: a left array, a center array, and a right array. When the needle crosses the imaging plane of the left array, the light source 802 can emit a green light. However, if the needle tip passes out of the imaging plane of the left array and crosses into the imaging plane of the center array, the light source 802 can change the color of the light emitted to red. In some embodiments, the color of the light emitted by the light source 802 corresponds to the array whose imaging plane the needle tip most recently crossed. For instance, green can indicate that the needle tip has crossed the imaging plane of the center array, red can indicate that the needle tip has crossed the imaging plane of the left array, and orange can indicate that the needle tip has crossed the imaging plane of the right array. Additionally or alternatively, the light source 802 can change its intensity and/or blinking pattern to indicate which array's imaging plane the needle tip has most recently crossed.

In some embodiments, the light source 802 includes multiple light sources spatially separated on the ultrasound scanner, such as side-by-side (not shown in FIG. 8 for clarity). Each of the arrays can correspond to one of the light sources. When the needle crosses the imaging plane of one of the arrays, the system can cause the light source that corresponds to that array to project light. Hence, the user can track the trajectory of the out-of-plane needle insertion by inspection of which light source is projecting light, and/or the color of the light, and/or the blinking pattern of the light.

### Example Transducer Arrays

In some embodiments, an ultrasound scanner, such as a wearable ultrasound scanner or a handheld ultrasound scanner, for determining port health with ultrasound includes a multi-array scanner (e.g., a multi-array transducer). A multi-array scanner in accordance with the present invention can include one or more of the arrays described in U.S. patent application Ser. No. 18/613,694, filed on Mar. 22, 2024, and entitled Multi-Dimensional and Multi-Frequency Ultrasound Transducers to Zhang et al., the disclosure of which is incorporated herein by reference in its entirety. In some embodiments, a multi-array scanner includes one or more of the arrays described in U.S. patent application Ser. No. 17/561,313, filed on Dec. 23, 2021, entitled Array Architecture and Interconnection for Transducers to Li et al., the disclosure of which is incorporated herein by reference in its entirety.

In some embodiments, a multi-array scanner for determining port health with ultrasound includes a first array with array elements selected from the group consisting of PZT, PMUT, and CMUT array elements, and a second array with additional array elements selected from the group consisting of PZT, PMUT, and CMUT array elements. The elements of the first array can be of a different type than the elements of the second array (e.g., the first array can include PMUT or PZT elements, and the second array can include CMUT elements).

In conventional PMUT and CMUT transducers, the vibration mode and operation frequency rely on the structure of the membrane size and thickness. Conventional CMUT transducers generally have a broader bandwidth with more uniform cell size than conventional PMUT arrays. However, the transmitting sensitivity (in terms of power efficiency) of conventional CMUT arrays is usually weaker than conventional PMUT arrays. On the other hand, conventional PMUT arrays usually have better transmitting sensitivity, but narrower bandwidth, than conventional CMUT arrays. To increase the bandwidth, some conventional PMUT arrays use different vibration cell sizes, e.g., different cells contribute acoustic energy with different operation frequencies to reach an overall broader bandwidth. However, this method reduces transmitting sensitivity, and can require significant tuning effort. Thus, conventional PMUT and CMUT arrays usually compromise performance between sensitivity and bandwidth, and may not be suitable for some applications, such as determining port health with ultrasound.

In contrast, systems, devices, and methods for determining port health with ultrasound, including multi-array transducers with combinations of PZT, PMUT, and CMUT arrays that can operate at low and high frequencies based not only on the mechanical structures, but also the electrical complex impedance tuning is disclosed. For example, in some embodiments, the multi-array transducers include one or more PMUT arrays and one or more CMUT arrays. In some embodiments, for each low or high frequency array, the system controls the arrays independently, which facilitates unique imaging modes, including super-harmonic imaging.

In some embodiments, the system includes a multi-section, multi-functional, multi-frequency transducer using multiple uniform vibration sections combined to reach high sensitivity and broader bandwidth. In each section, the cells can have the same mechanical structure. However, the cells can be different between each of the sections. For each PMUT, CMUT, or PZT section, the elements in each functional area can be tuned with the same inductors (e.g., tuning impedances). However, the tuning inductors between each section can be different depending on the desired performance of the overall transducers. The transducer's overall broader bandwidth compared to conventional transducers can be reached through multiple narrow band array sections with each section having a different operation frequency.

The PMUT, CMUT, and/or PZT sections can be tuned differently to enhance the performances. In each section, the cells can be uniformly constructed to provide optimized vibration, and therefore high sensitivity. The overall transducer bandwidth can be reached from combining several sections in the elevational direction, where each section can have a different operation frequency. Each section of the PMUT, CMUT, and/or PZT arrays can be electrically tuned differently to enhance both sensitivity and bandwidth.

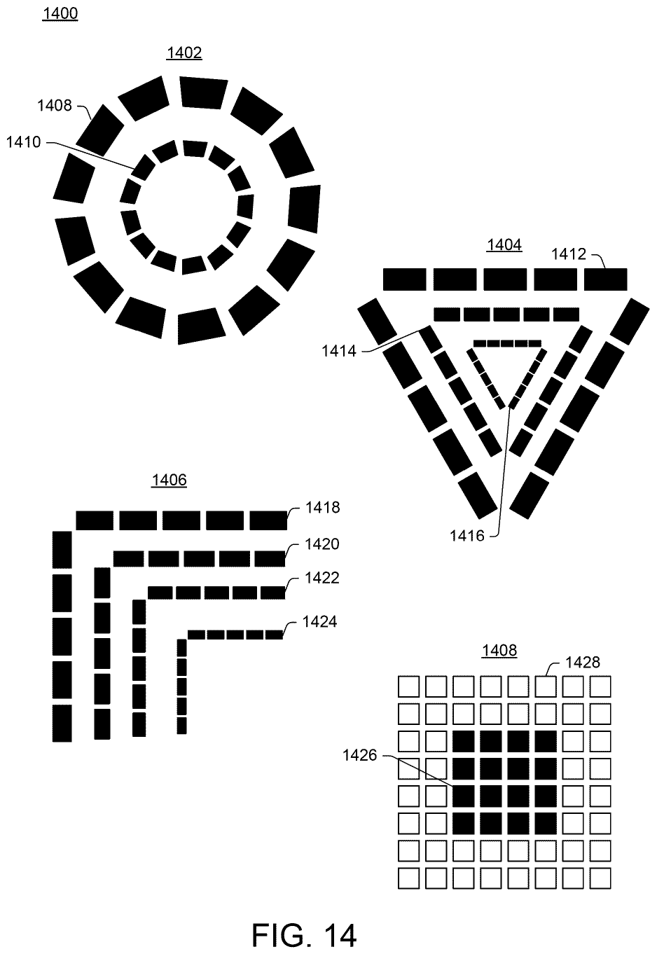

FIG. 9 illustrates some embodiments of a multi-array transducer 900 for determining port health with ultrasound. The multi-array transducer 900 includes three arrays, or sub-arrays, 902, 904, and 906. The first array 902 can be referred to as a center array, as it is between the second array 904 and the third array 906, which can be referred to as adjacent arrays. In this example multi-array transducer 900, the arrays 902-906 are laid out in rows, parallel to one another. As will be discussed below with respect to FIG. 14, multi-array transducers in accordance with the present invention are not so limited. The example multi-array transducer 900 also includes a lens 908 that covers the three arrays 902-906. In the example in FIG. 9, the lens 908 includes multiple radii of curvature. For example, a first radius covers the array 902, a second radius covers the array 904, and a third radius covers the array 906. In an example, the second radius and the third radius are the same radius, which is different than the first radius.

In the example in FIG. 9, the arrays 902-906 of the multi-array transducer 900 include PZT array elements. However, multi-array transducers in accordance with the present invention are not so limited and can include arrays in any suitable combination of PZT, PMUT, and CMUT array elements. In some embodiments, the array 902 can include PZT array elements, and the adjacent arrays 904 and 906 can include PMUT array elements. In another example, the array 902 can include PZT array elements, and the adjacent arrays 904 and 906 can include CMUT array elements. In some other embodiments, the array 902 can include PMUT array elements, and the adjacent arrays 904 and 906 can include CMUT array elements. In another example, the array 902 can include PMUT array elements, and the adjacent arrays 904 and 906 can include PZT array elements.

In some embodiments, the first array 902 operates at a first frequency, and the second and third arrays 904 and 906 operate at a second frequency that is different than the first frequency. For instance, the second frequency can be lower or higher than the first frequency. In some embodiments, the second and third arrays 904 and 906 operate at different frequencies from one another, which can be higher or lower than the first frequency. The frequencies of the arrays 902-906 can be selected so that the bandwidths of the arrays overlap, and so that the union of the individual bandwidths extends the overall bandwidth of the multi-array transducer 900.

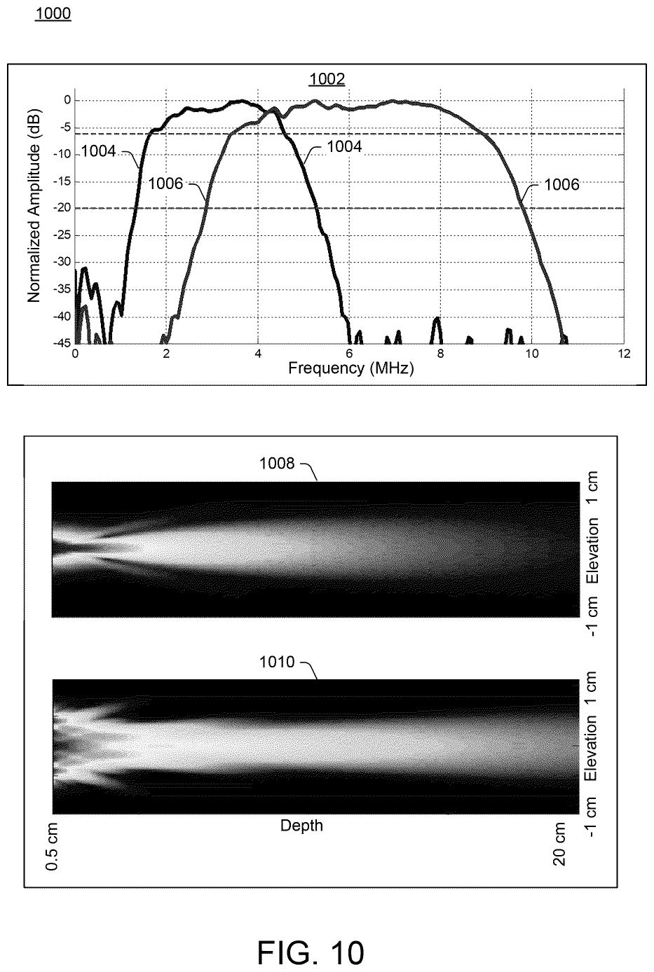

For example, FIG. 10 illustrates example characteristics 1000 of a multi-array transducer for determining port health with ultrasound. The characteristics 1000 include a frequency response 1002 of a multi-array transducer, such as the multi-array transducer 900 in FIG. 9. The frequency response 1002 includes a first bandwidth 1004 and a second bandwidth 1006. The first bandwidth 1004 illustrates the frequency response of an array, such as the arrays 904 and 906 in FIG. 9, and the second bandwidth 1006 illustrates the frequency response of another array, such as the array 902 in FIG. 9. By combining the first bandwidth 1004 and the second bandwidth 1006, the overall bandwidth of the multi-array transducer is increased.

The characteristics 1000 also include illustrations of a first ultrasound beam 1008 and a second ultrasound beam 1010 showing depth against elevation. The first ultrasound beam 1008 corresponds to the array 902 in FIG. 9, and the second ultrasound beam 1010 corresponds to the arrays 904 and 906 in FIG. 9. Because the array 902 is implemented to operate at a higher frequency than the arrays 904 and 906, the second ultrasound beam 1010 has deeper penetration than the first ultrasound beam 1008, but the first ultrasound beam 1008 has better focus than the second ultrasound beam 1010. Hence, the multi-array transducer can exploit the different ultrasound beam profiles associated with the multiple arrays to image at multiple depths with a same ultrasound scanner, rather than requiring the use of multiple ultrasound scanners.

Further, because the transducer includes multiple arrays, these arrays can be implemented to configure the transducer in one of multiple operation (e.g., imaging) modes, as is discussed below with respect to Table 1. Moreover, because the transducer can include multiple arrays of different types of array elements (e.g., PZT, PMUT, and CMUT), the strengths of each of the types of array elements can be exploited. For example, PMUT, which conventionally has better transmit sensitivity than CMUT, can be used for ultrasound transmission, while CMUT, which conventionally has better receive sensitivity than PMUT, can be used for ultrasound reception.

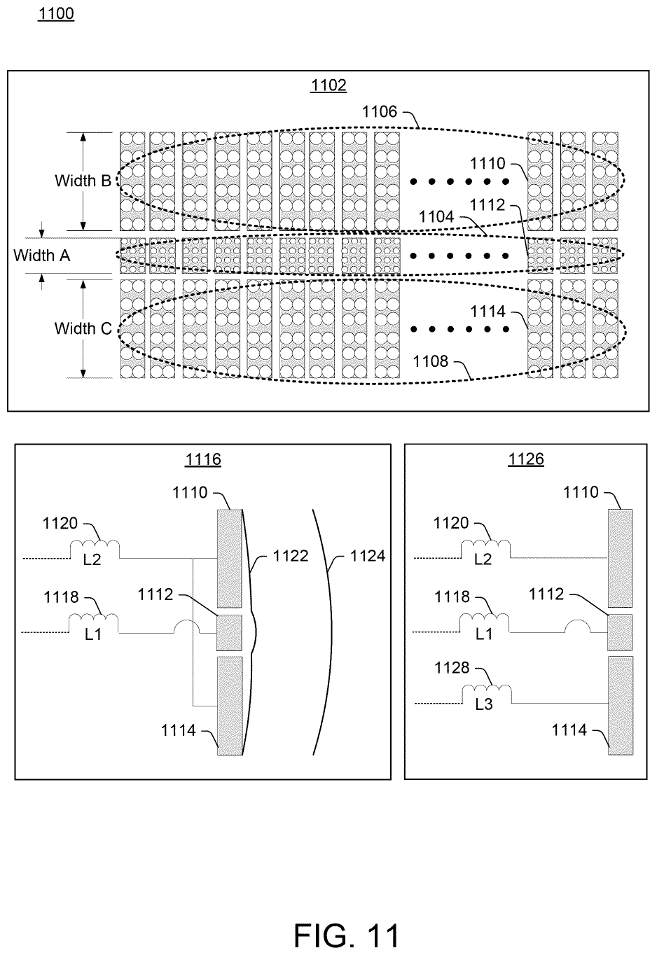

FIG. 11 illustrates some other embodiments of a multi-array transducer 1100 for determining port health with ultrasound. The multi-array transducer 1100 is an example of the multi-array transducer 1000. At inset 1102, the multi-array transducer 1100 includes a first array 1104 (e.g., a center array), and second and third arrays 1106 and 1108 (e.g., adjacent arrays). Each array includes multiple array elements, or sections. For example, section 1110 is an array element of the array 1106, section 1112 is an array element of the array 1104, and section 1114 is an array element of the array 1108. The sections of an array can include any type of array element. For instance, the arrays 1106 and 1108 can include CMUT array elements, and the array 1102 can include PMUT array elements. In another example, the arrays 1102-1106 can each be comprised of PMUT, CMUT, or PZT array elements. The array elements can be comprised of cells, which in this example are illustrated as circles for clarity. However, the cells can be of any suitable shape, such as ellipses, squares, rectangles, polygons, etc.

In the example multi-array transducer 1100, the array 1104 has a width of A, the array 1106 has a width of B, and the array 1108 has a width of C. In some embodiments, including when the arrays 1104 and 1106 (e.g., the adjacent arrays) are implemented to operate at a same frequency, the width B and the width C can be the same width. In some other embodiments, including when the arrays 1104 and 1106 are implemented to operate at different frequencies than one another, the width B can be different from the width C.

The array elements can be tuned to achieve a bandwidth for the array, and the tuning can include to couple a complex impedance to the array element. In some embodiments, each array element (or section) of an array is tuned with a same complex impedance. For instance, inset 1116 illustrates the sections 1110-1114 from the arrays 1104-1108, respectively. For clarity, the cells (circles) are omitted. Each of the sections 1110-1114 at inset 1116 are coupled to a complex impedance. For example, a complex impedance 1118 is coupled to the section 1112, and the complex impedance 1120 is coupled to both the sections 1110 and 1114. The sections 1110 and 1114 are coupled to the same complex impedance 1120 because in this example, width B and width C are equal, and the arrays 1106 and 1108 (e.g., the adjacent arrays) are implemented to operate at a same frequency as one another.

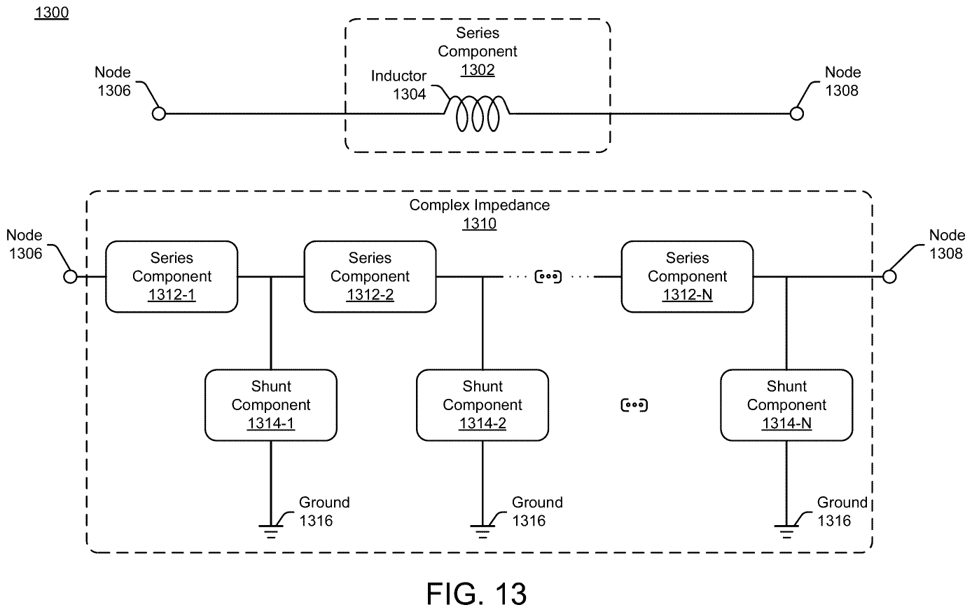

The complex impedances 1118 and 1120 in this example are illustrated for clarity as single inductors with values L1 and L2, respectively. However, as will become apparent below with regards to the discussion of FIG. 13, the complex impedances 1118 and 1120 are not limited to a single element or to just inductors, but can instead include any suitable combination and number of inductors, capacitors, and resistors in series and shunt configurations.

Also at inset 1116, the example multi-array transducer 1100 includes a lens 1122. The lens 1122 includes multiple radii of curvature and is an example of the lens 908. Additionally or alternatively, the multi-array transducer 1100 includes the lens 1124, which includes a single radius of curvature. A lens (e.g., the lens 1122 or the lens 1124) can cover the arrays 1104-1108.

In contrast to inset 1116 which illustrates an implementation of complex tuning impedances for the case when width B and width C are equal, the inset 1126 illustrates an implementation of complex tuning impedances for the case when width B and width C are not equal. In this case, the arrays 1106 and 1108 (e.g., the adjacent arrays) can be implemented to operate at different frequencies from one another. Accordingly, at inset 1126 each of the sections 1110, 1112, and 1114 are coupled to different complex impedances 1120, 1118, and 1128, respectively.

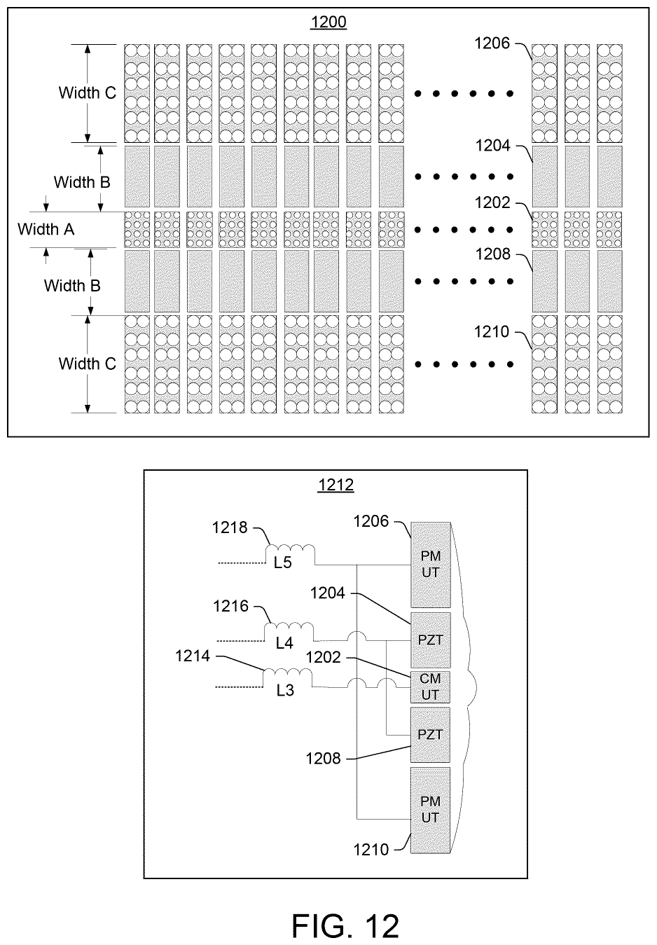

FIG. 12 illustrates some other embodiments of a multi-array transducer 1200 for determining port health with ultrasound. Referring to FIG. 12, the multi-array transducer 1200 is similar to the multi-array transducer 1100, but includes five arrays instead of three. For instance, the array element (or section) 1202 belongs to a center array having a width A. The array element (or section) 1204 belongs to an upper adjacent array having a width B. The array element (or section) 1206 belongs to an upper outer array having a width C. On the lower side of the center array, the array element (or section) 1208 belongs to a lower adjacent array having a width B, and the array element (or section) 1210 belongs to a lower outer array having a width C. In some embodiments, the center array of width B is configured to operate at a first frequency, the adjacent arrays of width B are configured to operate at a second frequency, and the outer arrays of width C are configured to operate at a third frequency. The second and third frequencies can be the same or different from one another and be lower or higher than the first frequency.

Inset 1212 illustrates an implementation of complex tuning impedances for the multi-array transducer 1200. The array elements of the center array, including the array element 1202, are coupled to a complex impedance 1214 (e.g., an inductor with value L3). The array elements of the adjacent arrays, including the array elements 1204 and 1208, are coupled to a complex impedance 1216 (e.g., an inductor with value LA). The array elements of the outer arrays, including the array elements 1206 and 1210, are coupled to a complex impedance 1218 (e.g., an inductor with value L5). As discussed below with respect to FIG. 13, the complex impedances 1214-1218 are not limited to a single element or to just inductors, but can instead include any suitable combination and number of inductors, capacitors, and resistors in series and shunt configurations.

In the example illustrated at the inset 1212, the multi-array transducer 1200 includes arrays comprised of PZT, PMUT, and CMUT array elements. For instance, the array elements of the center array, including the array element 1202, include CMUT array elements. The array elements of the adjacent arrays, including the array elements 1204 and 1208, include PZT array elements. The array elements of the outer arrays, including the array elements 1206 and 1210, include PMUT array elements. However, multi-array transducers in accordance with the present invention for use in determining port health can include any combination of arrays of PZT, PMUT, and/or CMUT array elements. For instance, the center array can include CMUT array elements, and both the adjacent arrays and the outer arrays can include PMUT array elements. In another example, the center array can include PZT array elements, upper arrays can include CMUT array elements, and lower arrays can include PMUT array elements.

FIG. 13 illustrates some embodiments of tuning impedances 1300 for transducer arrays for determining port health with ultrasound in accordance with the present invention. The tuning impedances 1300 include a series component 1302 that represents a complex impedance. In some embodiments, the series component 1302 includes a single inductor 1304 between the nodes 1306 and 1308. The inductor 1304 is an example of the inductors 1118, 1120, 1128, and 1214-1218.

The inductor 1304 is illustrated as an example circuit element, and generally the series component 1302 can include any suitable circuit element, such as inductor, capacitor, resistor, combinations thereof, and the like. Further, the series component 1302 can instead include any suitable combination and number of inductors, capacitors, resistors, or other series components in series and shunt configurations, as is illustrated by the complex impedance 1310.

In some embodiments, the complex impedance 1310 includes a series of series components 1312-1-1312-N between the nodes 1306 and 1308. In some embodiments, between each of the series components 1312-1-1312-N, the complex impedance 1310 includes one end of one of the shunt components 1314-1-1314-N. The other ends of the shunt components 1314-1-1314-N are connected to electrical ground 1316. Each of the series components 1312-1-1312-N and the shunt components 1314-1-1314-N can include any suitable circuit element, such as inductor, capacitor, resistor, combinations thereof, and the like. Accordingly, the complex impedance 1310 can be implemented to achieve any suitable tuning impedance for array elements of a multi-array transducer.

FIG. 14 illustrates some embodiments of an array configurations 1400 for multi-array transducers of ultrasound scanners for determining port health with ultrasound. The discussions of arrays of a multi-array scanner above largely focus on arrays comprised of rows of array elements, as illustrated in FIGS. 9, 11, and 12. However, the techniques disclosed herein are not limited to arrays (or sub-arrays) arranged in rows as previously described, but can also include multiple arrays in various configurations. For example, the example array configurations 1400 include multi-dimensional array architectures in accordance with some embodiments. The array configurations 1400 include a circular array configuration 1402, a polygonal array configuration 1404, an open-shaped array configuration 1406, and a matrix array configuration 1408. The arrays in the array configurations 1400 can include any suitable combination of PZT, PMUT, and CMUT array elements.

In some embodiments, the circular array configuration 1402 includes an outer array 1408 of transducer elements and an inner array 1410 of transducer elements arranged in concentric circles. Although circles are illustrated in the circular array configuration 1402, the outer array 1408 and the inner array 1410 can include elements arranged in concentric ellipses in some embodiments. Further, the circular array configuration 1402 can include more than the two concentric arrays that are illustrated. In one example, the inner array 1410 includes CMUT array elements, and the outer array 1408 includes PMUT array elements. In another example, the inner array 1410 includes PMUT array elements, and the outer array 1408 includes CMUT array elements.

In some embodiments, the polygonal array configuration 1404 includes three nested arrays of triangular shape, including an outer array 1412 of transducer elements, a center array 1414 of transducer elements, and an inner array 1416 of transducer elements. The triangular shapes of the three arrays of the polygonal array configuration 1404 are examples of polygons and are meant to be exemplary. Other polygonal shapes that can be included in the polygonal array 1404 include nested arrays arranged in rectangular, rhombus, pentagon, and the like shapes. In some embodiments, the center array 1414 includes CMUT array elements, and the outer array 1412 and the inner array 1416 include PMUT array elements. In another example, the center array 1414 includes PMUT array elements, and the outer array 1412 and the inner array 1416 include CMUT array elements.

In some embodiments, the open-shaped array configuration 1406 includes four nested arrays of L-shapes, including a first outer array 1418 of transducer elements, a second outer array 1420 of transducer elements, a first inner array 1422 of transducer elements, and a second inner array 1424 of transducer elements. The L-shapes of the four arrays of the open-shaped array 1406 are examples of open shapes and are meant to be exemplary. Other open shapes that can be included in the open-shaped array 1406 include nested arrays arranged in C-shapes, V-shapes, S-shapes, and the like. The first outer array 1418, second outer array 1420, first inner array 1422, and second inner array 1424 can include any suitable combination of PMUT, CMUT, and PZT array elements.

In some embodiments, the matrix array configuration 1408 includes an inner array 1426 having array elements on a grid, and an outer array 1428 having array elements on the grid and that surround the array elements of the inner array 1426. In an example, the inner array 1426 is centrally located within the outer array 1428. The inner array 1426 can include PMUT array elements that operate at a first frequency, and the outer array 1428 can include CMUT array elements that operate at a second frequency. In another example, the inner array 1426 can include CMUT array elements that operate at a third frequency, and the outer array 1428 can include PMUT array elements that operate at a fourth frequency. In some embodiments, the inner array 1426 operates at a higher frequency than the outer array 1428. In some embodiments, the inner array 1426 operates at a higher frequency than any other arrays of the matrix array configuration 1408.

In some embodiments, the matrix array configuration 1408 includes a third array (not shown for clarity) that surrounds the outer array 1428. The outer array 1428 can be centered within the third array. In another example, the matrix array configuration 1408 includes a fourth array (not shown for clarity) that surrounds the third array, and the third array can be centered within the fourth array. Hence, the matrix array configuration 1408 can include any suitable number of nested arrays. In an example, the matrix array configuration 1408 includes at least three arrays, including at least one PZT array, at least one PMUT array, and at least one CMUT array.

Table 1 illustrates operation (e.g., imaging) modes and transducer array configurations for a three-array transducer array, e.g., as is illustrated in FIG. 11. The center array represents the transducer array 1104, the first adjacent array represents the transducer array 1106, and the second adjacent array represents the transducer array 1108.

TABLE 1
Example operation (e.g., imaging) modes and transducer
array configurations for a three-array transducer array

| | Near Field | | | Far Field | | |
|---|---|---|---|---|---|---|
| | First | | Second | First | | Second |
| Operation | Adjacent | Center | Adjacent | Adjacent | Center | Adjacent |
| Mode | Array | Array | Array | Array | Array | Array |
| Mode 1 | Not Used | High Freq. | Not Used | Low Freq. | Not | Low Freq. |
| (Linear and | | (Tx/Rx) | | (Tx/Rx) | Used | (Tx/Rx) |
| Phased) | | (Linear) | | (Phased) | | (Phased) |
| Mode 2 | Low Freq. | Linear | Low Freq. | Phased | Linear | Phased |
| (Broadband | (Tx) | High Freq. | (Tx) | Low Freq. | High Freq. | Low Freq. |
| THI) | | (Tx/Rx) | | (Tx) | (Rx) | (Tx) |
| Mode 3 | Low Freq. | Linear | Low Freq. | Phased | Linear | Phased |
| (Full | (Tx/Rx) | High Freq. | (Tx/Rx) | Low Freq. | High Freq. | Low Freq. |
| Aperture | | (Tx/Rx) | | (Tx/Rx) | (Tx/Rx) | (Tx/Rx) |
| and | | | | | | |
| Broadband | | | | | | |
| THI) | | | | | | |

### Example Machine-Learned Model

Many of the aspects described herein can be implemented using a machine-learned model. For the purposes of this disclosure, a machine-learned model is any model that accepts an input, analyzes and/or processes the input based on an algorithm derived via machine-learning training, and provides an output. A machine-learned model can be conceptualized as a mathematical function of the following form:

$$f ( \hat{s} , θ ) = \hat{y} \qquad Equation ( 1 )$$

In Equation (1), the operator f represents the processing of the machine-learned model based on an input and providing an output. The term s represents a model input, such as ultrasound data. The model analyzes/processes the input s using parameters θ to generate output ŷ (e.g., object identification, object segmentation, object classification, etc.). Both ŝ and ŷ can be scalar values, matrices, vectors, or mathematical representations of phenomena such as categories, classifications, image characteristics, the images themselves, text, labels, or the like. The parameters θ can be any suitable mathematical operations, including but not limited to applications of weights and biases, filter coefficients, summations or other aggregations of data inputs, distribution parameters such as mean and variance in a Gaussian distribution, linear algebra-based operators, or other parameters, including combinations of different parameters, suitable to map data to a desired output.

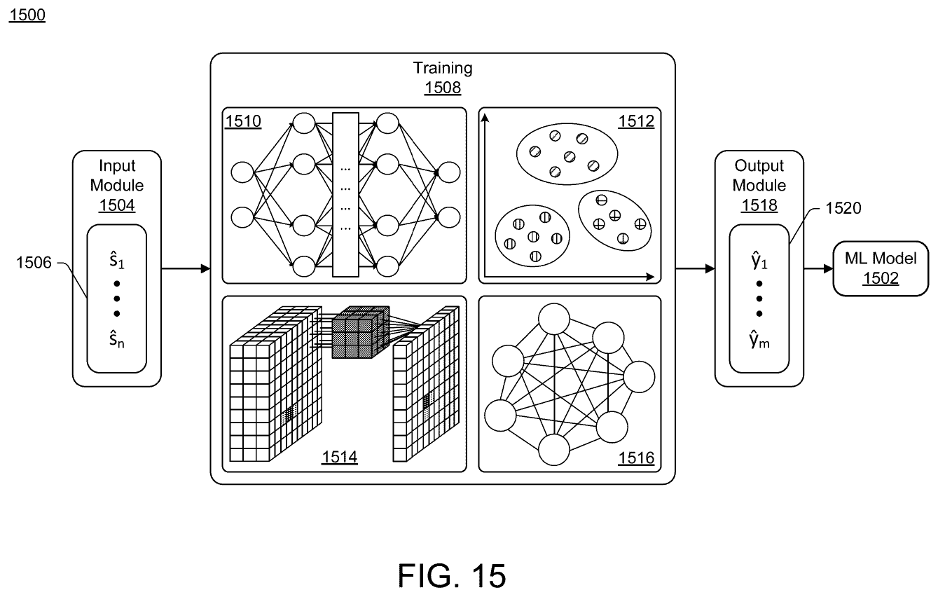

FIG. 15 represents some embodiments of a machine-learning architecture 1500 used to train a machine-learned model M 1502. An input module 1504 accepts an input ŝ 1506, which can be an array with members ŝ₁ through ŝₙ. The input ŝ 1506 is fed into a training module 1508, which processes the input ŝ 1506 based on the machine-learning architecture 1500. For example, if the machine-learning architecture 1500 uses a multilayer perceptron (MLP) model 1510, the training module 1508 applies weights and biases to the input ŝ 1506 through one or more layers of perceptrons, each perceptron performing a fit using its own weights and biases according to its given functional form. MLP weights and biases can be adjusted so that they are optimized against a least mean square, logcosh, or other optimization function (e.g., loss function) known in the art. Although an MLP model 1510 is described here as an example, any suitable machine-learning technique can be employed, some examples of which include but are not limited to k-means clustering 1512, convolutional neural networks (CNN) 1514, a Boltzmann machine 1516, Gaussian mixture models (GMM), and long short-term memory (LSTM). The training module 1508 provides an input to an output module 1518. The output module 1518 analyzes the input from the training module 1508 and provides an output in the form of ŷ 1520, which can be an array with members ŷ₁ through ŷₘ. The output 1520 can represent a known correlation with the input s 1506, such as, for example, object identification, segmentation, and/or classification.

In some examples, the input ŝ 1506 can be a training input labeled with known output correlation values, and these known values can be used to optimize the output ŷ 1520 in training against the optimization/loss function. In other examples, the machine-learning architecture 1500 can categorize the output ŷ 1520 values without being given known correlation values to the inputs ŝ 1506. In some examples, the machine-learning architecture 1500 can be a combination of machine-learning architectures. By way of example, a first network can use the input ŝ 1506 and provide the output ŷ 1520 as an input SML to a second machine-learned architecture, with the second machine-learned architecture providing a final output ŷ_{f}. In another example, one or more machine-learning architectures can be implemented at various points throughout the training module 1508.

In some machine-learned models, all layers of the model are fully connected. For example, all perceptrons in an MLP model act on every member of s. For an MLP model with a 100×100 pixel image as the input, each perceptron provides weights/biases for 10,000 inputs. With a large, densely layered model, this may result in slower processing and/or issues with vanishing and/or exploding gradients. A CNN, which may not be a fully connected model, can process the same image using 5×5 tiled regions, requiring only 25 perceptrons with shared weights, giving much greater efficiency than the fully connected MLP model.

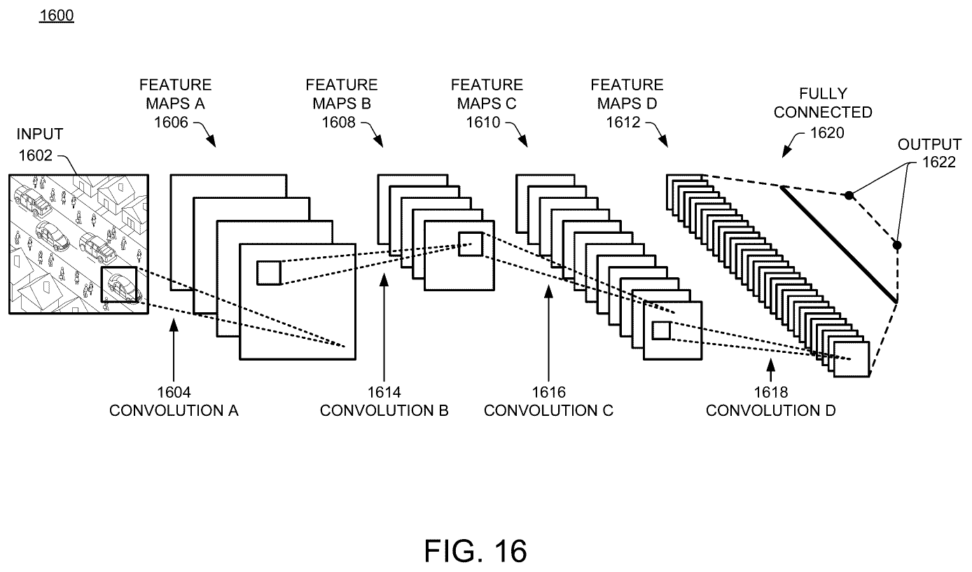

FIG. 16 represents some embodiments of a model 1600 using a CNN to process an input image 1602, which includes representations of objects that can be identified via object recognition, such as people or cars (or an anatomy, as described in relation to FIGS. 1-15). Convolution A 1604 can be performed to create a first set of feature maps (e.g., feature maps A 1606). A feature map can be a mapping of aspects of the input image 1602 given by a filter element of the CNN. This process can be repeated using feature maps A 1606 to generate further feature maps B 1608, feature maps C 1610, and feature maps D 1612 using convolution B 1614, convolution C 1616, and convolution D 1618, respectively. In this example, the feature maps D 1612 become an input for fully connected network layers 1620. In this way, the machine-learned model can be trained to recognize certain elements of the image, such as people, cars, or a particular patient anatomy, and provide an output 1622 that, for example, identifies the recognized elements. In some embodiments, an inference generated with an ultrasound system can be appended to a feature map (e.g., feature map B 1608) generated by a neural network (e.g., CNN). In this way, the feature vector and/or inference can be used as a secondary/conditional input to the neural network.

Although the example of FIG. 16 shows a CNN as a part of a fully connected network, other architectures are possible and this example should not be seen as limiting. There can be more or fewer layers in the CNN. A CNN component for a model can be placed in a different order, or the model can contain additional components or models. There may be no fully connected components, such as a fully convolutional network. Additional aspects of the CNN, such as pooling, downsampling, upsampling, or other aspects known to people skilled in the art can also be employed.

### Example Devices

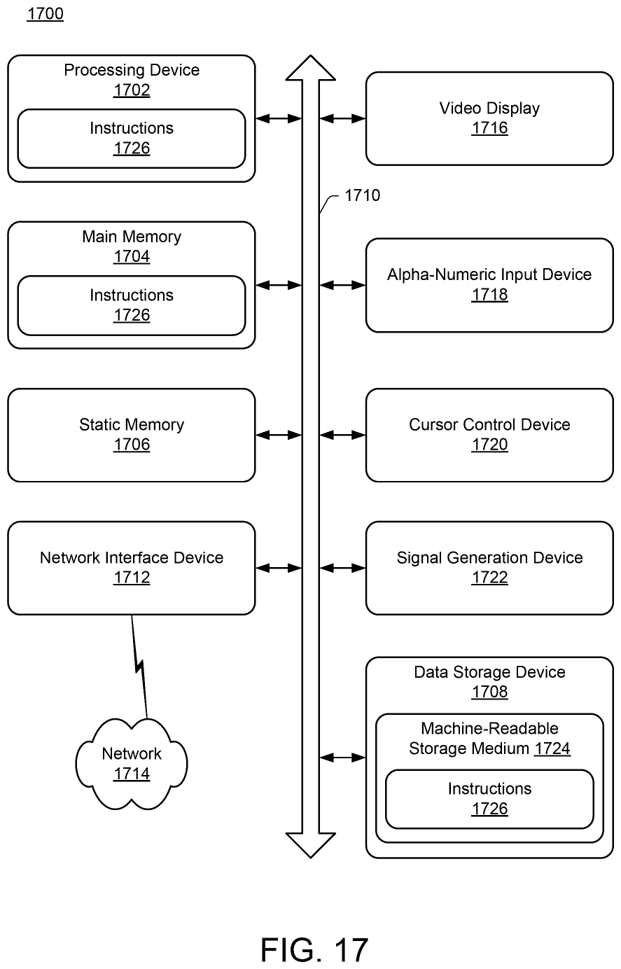

FIG. 17 illustrates a block diagram of some embodiments of a computing device 1700 that can perform one or more of the operations described herein, in accordance with some implementations. The computing device 1700 can be connected to other computing devices in a local area network (LAN), an intranet, an extranet, and/or the Internet. The computing device can operate in the capacity of a server machine in a client-server network environment or in the capacity of a client in a peer-to-peer network environment. The computing device can be provided by a personal computer (PC), a server computer, a desktop computer, a laptop computer, a tablet computer, a smartphone, an ultrasound machine, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein. In some implementations, the computing device 1700 is one or more of an ultrasound machine, an ultrasound scanner, an access point, a charging station, and a medical archiver.

The example computing device 1700 can include a processing device 1702 (e.g., a general-purpose processor, a programmable logic device (PLD), etc.), a main memory 1704 (e.g., synchronous dynamic random-access memory (DRAM), read-only memory (ROM), etc.), and a static memory 1706 (e.g., flash memory, a data storage device 1708, etc.), which can communicate with each other via a bus 1710. The processing device 1702 can be provided by one or more general-purpose processing devices such as a microprocessor, a central processing unit, or the like. In an illustrative example, the processing device 1702 comprises a complex instruction set computing (CISC) microprocessor, a reduced instruction set computing (RISC) microprocessor, a very long instruction word (VLIW) microprocessor, or a processor implementing other instruction sets or processors implementing a combination of instruction sets. The processing device 1702 can also comprise one or more special-purpose processing devices such as an application-specific integrated circuit (ASIC), a field programmable gate array (FPGA), a digital signal processor (DSP), a network processor, or the like. The processing device 1702 can be configured to execute the operations described herein, in accordance with one or more aspects of the present disclosure, for performing the operations and steps discussed herein.

The computing device 1700 can further include a network interface device 1712, which can communicate with a network 1714. The computing device 1700 also can include a video display unit 1716 (e.g., a liquid crystal display (LCD), an organic light-emitting diode (OLED), a cathode ray tube (CRT), etc.), an alphanumeric input device 1718 (e.g., a keyboard), a cursor control device 1720 (e.g., a mouse), and an acoustic signal generation device 1722 (e.g., a speaker, a microphone, etc.). In one embodiment, the video display unit 1716, the alphanumeric input device 1718, and the cursor control device 1720 can be combined into a single component or device (e.g., an LCD touch screen).

The data storage device 1708 can include a computer-readable storage medium 1724 on which can be stored one or more sets of instructions 1726 (e.g., instructions for carrying out the operations described herein, in accordance with one or more aspects of the present disclosure). The instructions 1726 can also reside, completely or at least partially, within the main memory 1704 and/or within the processing device 1702 during execution thereof by the computing device 1700, where the main memory 1704 and the processing device 1702 also constitute computer-readable media. The instructions can further be transmitted or received over the network 1714 via the network interface device 1712.

Various techniques are described in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. In some aspects, the modules described herein are embodied in the data storage device 1708 of the computing device 1700 as executable instructions or code. Although represented as software implementations, the described modules can be implemented as any form of a control application, software application, signal processing and control module, hardware, or firmware installed on the computing device 1700.

While the computer-readable storage medium 1724 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding, or carrying a set of instructions for execution by the machine and that causes the machine to perform the methods described herein. The term “computer-readable storage medium” shall accordingly be taken to include, but not be limited to, solid-state memories, optical media, and magnetic media.

### Example Procedures

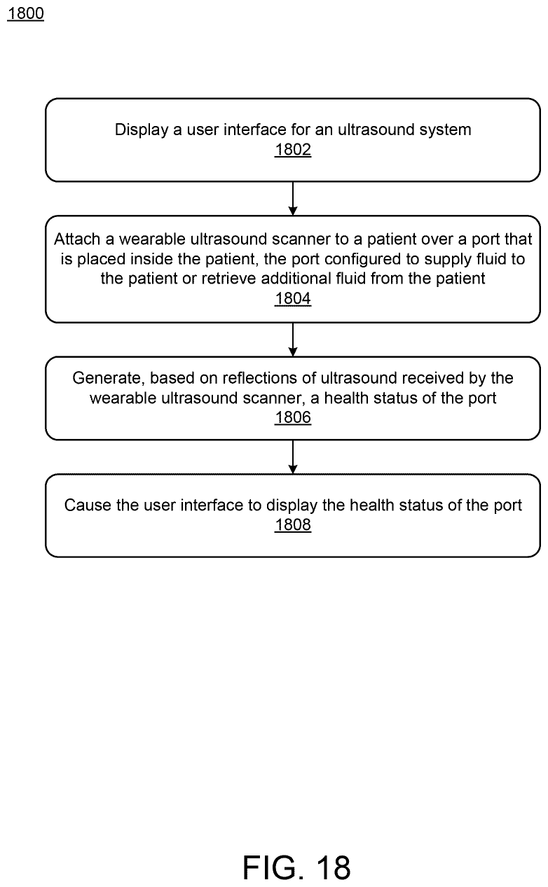

FIG. 18 illustrates some embodiments of a method 1800 that can be implemented by an ultrasound system (e.g., the ultrasound system in the environment 100, the ultrasound system in the implementation 200, and the ultrasound system in the environment 300) for determining port health with ultrasound. The ultrasound system can include an ultrasound scanner (e.g., transducer or probe), an ultrasound machine, a processor system, and a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 17.

A user interface for an ultrasound system is displayed (block 1802). For instance, a display device (e.g., the display device 108 and/or the display 412) can display the user interface. A wearable ultrasound scanner is attached to a patient over a port that is placed inside the patient, the port configured to supply fluid to the patient or retrieve additional fluid from the patient (block 1804). Based on reflections of ultrasound received by the wearable ultrasound scanner, a health status of the port is generated (block 1806). The user interface is caused to display the health status of the port (block 1808).

In some embodiments, the processor system generates the health status of the port including a prediction of when the port will fail. The prediction can include a number of days, hours, months, etc. that represents an expected time to failure for the port.

In some embodiments, the system includes a transceiver. The processor system can cause the transmitter to communicate, over a network, the health status to the display device to cause the user interface to display the health status. Additionally or alternatively, the processor system can determine, based on the health status of the port, transmission times. The transmission times can include periodic times, such as every 24 hours, times that are not periodic, such as in one day, three days, and five days. The processor system can generate additional health statuses of the port, and communicate, at the transmission times, the additional health statuses over the network to at least one of the display device and a medical archiver.

In aspects of determining port health with ultrasound, in some embodiments, the processor system generates the health status of the port based on at least one of an indication of infection of tissue proximate to the port, an amount of swelling of the tissue, an indication of congestion in the port, a measure of volume flow of the fluid or the additional fluid, a measure of the temperature of tissue proximate to the port, and a measure of pressure of the fluid or the additional fluid. The processor can implement a machine-learned model to generate the health status of the port. The machine-learned model can include a convolutional neural network, and one or more of the indication of infection of tissue proximate to the port, the amount of swelling of the tissue, the indication of congestion in the port, the measure of volume flow of the fluid or the additional fluid, a measure of the temperature of tissue proximate to the port, and the measure of pressure of the fluid or the additional fluid can be concatenated into an input vector that is processed by the convolutional neural network. Alternatively, some of these parameters can be can be concatenated into an input vector that is processed by layers of the convolutional neural network. The others of these parameters can be provided as conditional inputs to the convolutional neural network and concatenated with a feature vector that the convolutional neural network generates at the output of said layers of the convolutional neural network. The resulting concatenated vector can be processed by additional layers of the convolutional neural network.

In some embodiments, the wearable ultrasound scanner includes a patch that includes the processor system and a coupling agent implemented to couple the ultrasound from the wearable ultrasound scanner to the patient and couple the reflections from the patient to the wearable ultrasound scanner. The wearable ultrasound scanner can include a first transducer array and a second transducer array. The first transducer array can be implemented to transmit the ultrasound and the second transducer array can be implemented to receive the reflections of the ultrasound. In some embodiments, the first transducer array includes at least one of piezoelectric micromachined ultrasonic transducer (PMUT) array elements and lead zirconate titanate (PZT) array elements, and the second transducer array includes capacitive micromachined ultrasonic transducer (CMUT) array elements. The first transducer array can be implemented to operate at a first ultrasound frequency and the second transducer array can be implemented to operate at a second ultrasound frequency that is different from the first ultrasound frequency. In some embodiments, the first transducer array and the second transducer array are removably attached to the patch so that the first transducer array and the second transducer array can be removed and reattached to the patch at different positions on the patch.

In some embodiments, the wearable ultrasound scanner includes a patch and the display device is implemented to be removably attached to the patch. The patch can be disposable and the display device can be sterilized for reuse.

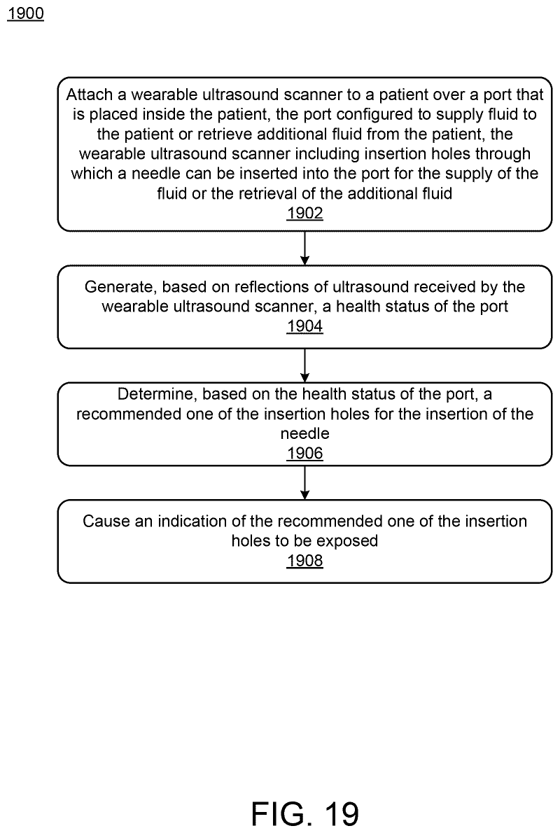

FIG. 19 illustrates some embodiments of a method 1900 that can be implemented by an ultrasound system (e.g., the ultrasound system in the environment 100, the ultrasound system in the implementation 200, and the ultrasound system in the environment 300) for determining port health with ultrasound. The ultrasound system can include an ultrasound scanner (e.g., transducer or probe), an ultrasound machine, a processor system, and a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 17.

A wearable ultrasound scanner is attached to a patient over a port that is placed inside the patient, where the port configured to supply fluid to the patient or retrieve additional fluid from the patient, the wearable ultrasound scanner including insertion holes through which a needle can be inserted into the port for the supply of the fluid or the retrieval of the additional fluid (block 1902). Based on reflections of ultrasound received by the wearable ultrasound scanner, a health status of the port is generated (block 1904). Based on the health status of the port, a recommended one of the insertion holes for the insertion of the needle is determined (block 1906). An indication of the recommended one of the insertion holes is caused to be exposed (block 1908).

In some embodiments, the wearable ultrasound scanner includes light sources proximate to the insertion holes. The exposure of the indication of the recommended one of the insertion holes can include to activate a light source of the light sources that is proximate to the recommended one of the insertion holes.

In some embodiments, the system includes a display device implemented to display the indication of the recommended one of the insertion holes. The wearable ultrasound scanner can include the display device. In some embodiments, the display device can display an orientation for the needle for the insertion through the recommended one of the insertion holes.

The processor system can generate the health status of the port based on at least one of an indication of infection of tissue proximate to the port, an amount of swelling of the tissue, an indication of congestion in the port, a measure of volume flow of the fluid or the additional fluid, a measure of the temperature of tissue proximate to the port, and a measure of pressure of the fluid or the additional fluid. For instance, the processor system can implement one or more machine-learned models that process these parameters as an input vector at a top layer of a CNN and/or as an input vector at the top layer and a conditional input vector input at a subsequent layer of the CNN, as previously described.

In some embodiments, the processor system is implemented to track, based on the reflections of the ultrasound, a tip of the needle. The processor system can then indicate, via a light source on the wearable ultrasound scanner, a current position of the tip of the needle. In some embodiments, the wearable ultrasound scanner includes multiple transducer arrays, and the processor system can determine one transducer array of the multiple transducer arrays that covers the current position of the tip of the needle, and select, based on the one transducer array, the light source from among multiple light sources on the wearable ultrasound scanner. In an example, the wearable ultrasound scanner includes multiple transducer arrays, and the processor system is implemented to determine one transducer array of the multiple transducer arrays that covers the current position of the tip of the needle, and select, based on the one transducer array, a color or lighting pattern of the light source.

In some embodiments, the processor system determines, based on the health status of the port, an additional one of the insertion holes not recommended to use for the insertion of the needle. The processor system can cause to be exposed an indication that the additional one of the insertion holes is not recommended for the insertion of the needle. For instance, the processor system can cause the indication to be exposed on a user interface of a display device, such as an ultrasound machine or the wearable ultrasound scanner.

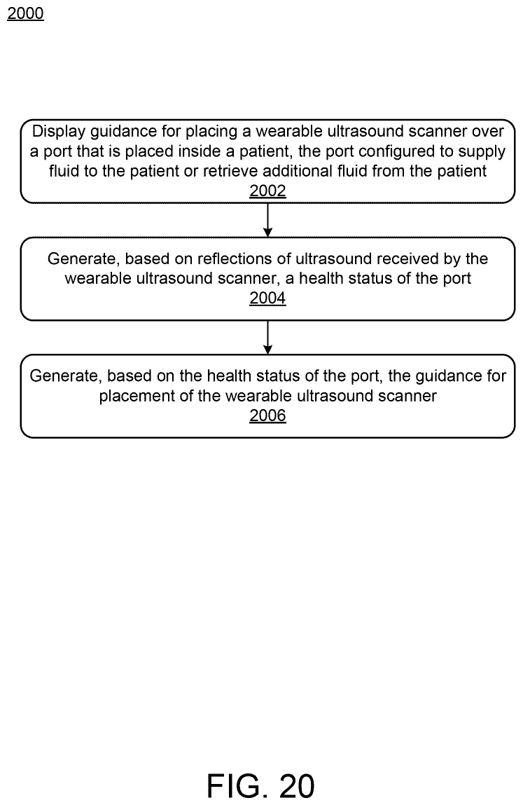

FIG. 20 illustrates some embodiments of a method 2000 that can be implemented by an ultrasound system (e.g., the ultrasound system in the environment 100, the ultrasound system in the implementation 200, and the ultrasound system in the environment 300) for determining port health with ultrasound. The ultrasound system can include an ultrasound scanner (e.g., transducer or probe), an ultrasound machine, a processor system, and a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 17.

Guidance for placing a wearable ultrasound scanner over a port that is placed inside a patient is displayed, the port configured to supply fluid to the patient or retrieve additional fluid from the patient (block 2002). Based on reflections of ultrasound received by a wearable ultrasound scanner, a health status of the port is generated (block 2004). Based on the health status of the port, the guidance for placement of the wearable ultrasound scanner is generated (block 2006).

In some embodiments, the processor system generates the health status of the port based on at least one of an indication of infection of tissue proximate to the port, an amount of swelling of the tissue, an indication of congestion in the port, a measure of volume flow of the fluid or the additional fluid, a measure of the temperature of tissue proximate to the port, and a measure of pressure of the fluid or the additional fluid. For instance, the processor system can implement one or more machine-learned models that process these parameters as an input vector at a top layer of a CNN and/or as an input vector at the top layer and a conditional input vector input at a subsequent layer of the CNN, as previously described.

In some embodiments, the system includes a transceiver implemented to transmit the health status of the port to a nurse station of a care facility. Additionally or alternatively, the transceiver can transmit the health status of the port to a server system maintained by the care facility. Additionally or alternatively, the transceiver can transmit the health status of the port to a medical archiver. Additionally or alternatively, the transceiver can transmit the health status of the port to an ultrasound machine, e.g., a point-of-care ultrasound (POCUS) ultrasound machine. Additionally or alternatively, the transceiver can transmit the health status of the port to a computing device that displays the guidance.

In some embodiments, the fluid includes at least one of a drug, saline fluid, dextrose fluid, lactated Ringer's fluid, and blood. Additionally or alternatively, the additional fluid can include at least one of blood, urine, extracellular fluid, semen, amniotic fluid, cerebrospinal fluid, and plasma.

In aspects of determining port health with ultrasound, in some embodiments, the guidance includes a location and an orientation of the wearable ultrasound scanner, the location and the orientation relative to the port. Additionally or alternatively, the guidance can include an image that depicts a view that should be obtained by the wearable ultrasound device when properly placed. Additionally or alternatively, the wearable ultrasound scanner can include multiple transducer arrays, and the processor system can select, based on the health status of the port, one of the multiple transducer arrays. The processor system can then determine at least one of the location and the orientation so that the one of the multiple transducer arrays is positioned over the port.

In some embodiments, the processor system can, after the wearable ultrasound scanner is placed on the patient based on the guidance, generate, based on additional reflections of ultrasound received by the wearable ultrasound scanner, an additional health status of the port. The additional health status of the port can include an expected time to failure for the port. Additionally or alternatively, the processor system can schedule, based on the expected time to failure, an appointment for the patient for replacement of the port. Additionally or alternatively, the processor system can cause the display device to display a recommendation, based on the expected time to failure, to schedule an appointment for the patient for replacement of the port.

There are a number of example embodiments described herein.

Example 1 is an ultrasound system including a display device configured to display a user interface for the ultrasound system and a wearable ultrasound scanner configured to be attached to a patient over a port that is placed inside the patient, where the port is configured to supply fluid to the patient or retrieve additional fluid from the patient. The ultrasound system also includes a processor system configured to generate a health status of the port based on reflections of ultrasound received by the wearable ultrasound scanner and cause the user interface to display the health status of the port.

Example 2 is the ultrasound system of example 1 that may optionally include that the processor system is implemented to generate the health status of the port including a prediction of when the port will fail.

Example 3 is the ultrasound system of example 1 that may optionally include a transceiver, wherein the processor system is implemented to cause the transmitter to communicate, over a network, the health status to the display device to display of the health status on the user interface.

Example 4 is the ultrasound system of example 3 that may optionally include that the processor system is implemented to: determine, based on the health status of the port, transmission times, generate additional health statuses of the port, and communicate, at the transmission times, at least one of the additional health statuses over the network to at least one of the display device and a medical archiver.

Example 5 is the ultrasound system of example 1 that may optionally include that the processor system is implemented to generate the health status of the port based on at least one of an indication of infection of tissue proximate to the port, an amount of swelling of the tissue, an indication of congestion in the port, a measure of volume flow of the fluid or the additional fluid, a measure of the temperature of the tissue proximate to the port, and a measure of pressure of the fluid or the additional fluid.

Example 6 is the ultrasound system of example 5 that may optionally include that the processor system is configured to run a machine-learned model to generate the health status of the port.

Example 7 is the ultrasound system of example 1 that may optionally include that the wearable ultrasound scanner includes a patch that includes the processor system and a coupling agent implemented to couple the ultrasound from the wearable ultrasound scanner to the patient and couple the reflections from the patient to the wearable ultrasound scanner.

Example 8 is the ultrasound system of example 7 that may optionally include that the wearable ultrasound scanner includes a first transducer array and a second transducer array, where the first transducer array is implemented to transmit the ultrasound and the second transducer array implemented to receive the reflections of the ultrasound.

Example 9 is the ultrasound system of example 8 that may optionally include that the first transducer array includes at least one of piezoelectric micromachined ultrasonic transducer (PMUT) array elements and lead zirconate titanate (PZT) array elements, and the second transducer array includes capacitive micromachined ultrasonic transducer (CMUT) array elements.

Example 10 is the ultrasound system of example 9 that may optionally include that the first transducer array is implemented to operate at a first ultrasound frequency and the second transducer array is implemented to operate at a second ultrasound frequency that is different from the first ultrasound frequency.

Example 11 is the ultrasound system of example 8 that may optionally include that the first transducer array and the second transducer array are removably attached to the patch so that the first transducer array and the second transducer array can be removed and reattached to the patch at different positions on the patch.

Example 12 is the ultrasound system of example 1 that may optionally include that the wearable ultrasound scanner includes a patch and the display device is removably attachable to the patch.

Example 13 is the ultrasound system of example 12 that may optionally include that the patch is disposable and the display device can be sterilized for reuse.

Example 14 is an ultrasound system including a wearable ultrasound scanner configured to be attached to a patient over a port that is placed inside the patient, the port configured to supply fluid to the patient or retrieve additional fluid from the patient, wherein the wearable ultrasound scanner includes insertion holes through which a needle can be inserted into the port for the supply of the fluid or the retrieval of the additional fluid. The ultrasound system also includes a processor system configured to generate, based on reflections of ultrasound received by the wearable ultrasound scanner, a health status of the port, determine, based on the health status of the port, a recommended one of the insertion holes for the insertion of the needle, and cause an indication of the recommended one of the insertion holes to be exposed.

Example 15 is the ultrasound system of example 14 that may optionally include that the wearable ultrasound scanner includes light sources proximate to the insertion holes, and exposure of the indication of the recommended one of the insertion holes includes activating a light source of the light sources that is proximate to the recommended one of the insertion holes.

Example 16 is the ultrasound system of example 14 that may optionally include that a display device implemented to display the indication of the recommended one of the insertion holes.

Example 17 is the ultrasound system of example 16 that may optionally include that the wearable ultrasound scanner includes the display device.

Example 18 is the ultrasound system of example 16 that may optionally include that the display device is implemented to display an orientation for the needle for the insertion through the recommended one of the insertion holes.

Example 19 is the ultrasound system of example 14 that may optionally include that the processor system is implemented to generate the health status of the port based on at least one of an indication of infection of tissue proximate to the port, an amount of swelling of the tissue, an indication of congestion in the port, a measure of volume flow of the fluid or the additional fluid, a measure of the temperature of the tissue proximate to the port, and a measure of pressure of the fluid or the additional fluid.

Example 20 is the ultrasound system of example 14 that may optionally include that the processor system is implemented to: track, based on the reflections of the ultrasound, a tip of the needle and indicate, via a light source on the wearable ultrasound scanner, a current position of the tip of the needle.

Example 21 is the ultrasound system of example 20 that may optionally include that the wearable ultrasound scanner includes multiple transducer arrays, and the processor system is implemented to determine one transducer array of the multiple transducer arrays that covers the current position of the tip of the needle and select, based on the one transducer array, the light source from among multiple light sources on the wearable ultrasound scanner.

Example 22 is the ultrasound system of example 20 that may optionally include that the wearable ultrasound scanner includes multiple transducer arrays, and the processor system is implemented to determine one transducer array of the multiple transducer arrays that covers the current position of the tip of the needle and select, based on the one transducer array, a color or lighting pattern of the light source.

Example 23 is the ultrasound system of example 14 that may optionally include that the processor system is implemented to determine, based on the health status of the port, an additional one of the insertion holes not recommended to use for the insertion of the needle, and cause to be exposed an indication that the additional one of the insertion holes is not recommended for the insertion of the needle.

Example 24 is an ultrasound system including a wearable ultrasound scanner and a display device configured to display guidance for placing a wearable ultrasound scanner over a port that is placed inside a patient, where the port configured to supply fluid to the patient or retrieve additional fluid from the patient. The ultrasound system also including a processor system configured to generate, based on reflections of ultrasound received by the wearable ultrasound scanner, a health status of the port, and generate, based on the health status of the port, the guidance for placement of the wearable ultrasound scanner.

Example 25 is the ultrasound system of example 24 that may optionally include that the processor system is implemented to generate the health status of the port based on at least one of an indication of infection of tissue proximate to the port, an amount of swelling of the tissue, an indication of congestion in the port, a measure of volume flow of the fluid or the additional fluid, and a measure of pressure of the fluid or the additional fluid.

Example 26 is the ultrasound system of example 24 that may optionally include a transceiver implemented to transmit the health status of the port to a nurse station of a care facility.

Example 27 is the ultrasound system of example 24 that may optionally include that the fluid includes at least one of a drug, saline fluid, dextrose fluid, lactated Ringer's fluid, and blood.

Example 28 is the ultrasound system of example 24 that may optionally include that the additional fluid includes at least one of blood, urine, extracellular fluid, semen, amniotic fluid, cerebrospinal fluid, and plasma.

Example 29 is the ultrasound system of example 24 that may optionally include that the guidance includes a location and an orientation of the wearable ultrasound scanner, the location and the orientation being relative to the port.

Example 30 is the ultrasound system of example 29 that may optionally include that the wearable ultrasound scanner includes multiple transducer arrays, and the processor system is implemented to select, based on the health status of the port, one of the multiple transducer arrays, and determine at least one of the location and the orientation so that the one of the multiple transducer arrays is positioned over the port.

Example 30 is the ultrasound system of example 24 that may optionally include that the processor system is implemented to, after the wearable ultrasound scanner is placed on the patient based on the guidance, generate, based on additional reflections of ultrasound received by the wearable ultrasound scanner, an additional health status of the port.

Example 31 is the ultrasound system of example 30 that may optionally include that the additional health status of the port includes an expected time to failure for the port.

Example 32 is the ultrasound system of example 31 that may optionally include that the processor system is implemented to schedule, based on the expected time to failure, an appointment for the patient for replacement of the port.

Example 33 is the ultrasound system of example 31 that may optionally include that the processor system is implemented to cause the display device to display a recommendation, based on the expected time to failure, to schedule an appointment for the patient for replacement of the port.

Example 34 includes an ultrasound scanner comprising a first transducer array including first sections of first array elements having a first width, the first array elements being of a first element type; a second transducer array including second sections of second array elements having a second width that is different than the first width, the second array elements being of a second element type that is different than the first element type; and a lens having a first radius of curvature that covers the first transducer array and a second radius of curvature that covers the second transducer array.

Example 35 includes the ultrasound scanner as described in example 34, wherein the first sections of the first array elements are arranged in a first row, and the second sections of the second array elements are arranged in a second row that is parallel to the first row.

Example 36 includes the ultrasound scanner as described in example 35, further comprising a third transducer array including third sections of third array elements having the second width, wherein the third sections of the third array elements are arranged in a third row that is parallel to the first row and on an opposite side of the first row than the second sections of the second array elements.

Example 37 includes the ultrasound scanner as described in example 34, wherein the first element type includes piezoelectric micromachined ultrasonic transducer (PMUT) array elements, and the second element type includes capacitive micromachined ultrasonic transducer (CMUT) array elements.

Example 38 includes the ultrasound scanner as described in example 37, wherein the PMUT array elements are implemented to transmit ultrasound and the CMUT array elements are implemented to receive reflections of the ultrasound.

Example 39 includes the ultrasound scanner as described in example 34, wherein the first element type includes lead zirconate titanate (PZT) array elements and the second element type includes capacitive micromachined ultrasonic transducer (CMUT) array elements.

Example 40 includes the ultrasound scanner as described in example 39, wherein the PZT array elements are implemented to transmit ultrasound and the CMUT array elements are implemented to receive reflections of the ultrasound.

Example 41 includes the ultrasound scanner as described in example 34, wherein the first element type includes piezoelectric micromachined ultrasonic transducer (PMUT) array elements and the second element type includes lead zirconate titanate (PZT) array elements.

Example 42 includes the ultrasound scanner as described in example 41, wherein one of the PMUT array elements and the PZT array elements is implemented to transmit ultrasound and the other of the PMUT array elements and the PZT array elements is implemented to receive reflections of the ultrasound.

Example 43 includes the ultrasound scanner as described in example 34, wherein the first array elements are coupled to complex impedances of a first tuning configuration and the second array elements are coupled to complex impedances of a second tuning configuration, the first tuning configuration being different from the second tuning configuration.

Example 44 includes the ultrasound scanner as described in example 43, wherein the first tuning configuration includes an inductor of a first value and the second tuning configuration includes an inductor of a second value that is different than the first value.

Example 45 includes the ultrasound scanner as described in example 43, wherein the first tuning configuration includes a different number of inductors than the second tuning configuration.

Example 46 includes the ultrasound scanner as described in example 43, wherein the first tuning configuration includes a different number of capacitors than the second tuning configuration.

Example 47 includes the ultrasound scanner as described in example 34, wherein the first transducer array and the second transducer array are implemented as a wearable ultrasound scanner configured to be attached to a patient over a port that is placed inside the patient, the port configured to supply fluid to the patient or retrieve additional fluid from the patient.

Example 48 includes the ultrasound scanner as described in example 47, further comprising a processor system implemented to generate, based on reflections of ultrasound received by the wearable ultrasound scanner, a health status of the port.

Examples of an ultrasound system that can include transducer arrays disclosed herein are described in Examples 49-53 below.

Example 49 includes an ultrasound system comprising: an ultrasound scanner including: a first transducer array including first sections of first array elements having a first width, the first array elements being of a first element type selected from the group consisting of piezoelectric micromachined ultrasonic transducer (PMUT) array elements, lead zirconate titanate (PZT) array elements, and capacitive micromachined ultrasonic transducer (CMUT) array elements; and a second transducer array including second sections of second array elements having a second width that is different than the first width, the second array elements being of a second element type that is different than the first element type and selected from the group consisting of the PMUT array elements, the PZT array elements, and the CMUT array elements; and a processor system configured to: determine an operation mode for the ultrasound system; configure, based on the operation mode, the first array elements to transmit ultrasound at a patient anatomy; and configure, based on the operation mode, the second array elements to receive reflections of the ultrasound from the patient anatomy.

Example 50 includes the ultrasound system as described in example 49, wherein the processor system is configured to: implement a machine-learned model to determine, based on an ultrasound image generated based on additional ultrasound transmitted by the ultrasound scanner, a classification of the patient anatomy; and determine, based on the classification of the patient anatomy, the operation mode.

Example 51 includes the ultrasound system as described in example 49, wherein the processor system is configured to: implement an ultrasound protocol; and determine, based on a current step of the ultrasound protocol, the operation mode.

Example 52 includes the ultrasound system as described in example 49, wherein the operation mode is selected from the group consisting of an imaging-on-overlap-bandwidth mode, a tissue-harmonic-imaging mode, and a full-aperture mode.

Example 53 includes the ultrasound system as described in example 49, wherein the processor system is implemented to configure, based on the operation mode, the first array elements to receive the reflections of the ultrasound from the patient anatomy.

Examples of an ultrasound system that can include transducer arrays disclosed herein are described in Examples 54-62 below.

Example 54 include an ultrasound system comprising: an ultrasound scanner including: a first transducer array including first sections of first array elements having a first width, the first array elements being of a first element type selected from the group consisting of piezoelectric micromachined ultrasonic transducer (PMUT) array elements, lead zirconate titanate (PZT) array elements, and capacitive micromachined ultrasonic transducer (CMUT) array elements; and a second transducer array including second sections of second array elements having a second width that is different than the first width, the second array elements being of a second element type selected from the group consisting of the PMUT array elements, the PZT array elements, and the CMUT array elements; and a processor system configured to: configure the first array elements to transmit ultrasound at a patient anatomy; and configure the second array elements to transmit additional ultrasound at an interventional instrument.

Example 55 includes the ultrasound system as described in example 54, wherein the first element type and the second element type are the same element type.

Example 56 includes the ultrasound system as described in example 54, wherein the first element type and the second element type are different element types.

Example 57 includes the ultrasound system as described in example 54, wherein the ultrasound scanner includes a light source, wherein the processor system is implemented to cause the light source to display light when the processor system determines a presence of the interventional instrument based on the additional ultrasound.

Example 58 includes the ultrasound system as described in example 57, wherein the processor system is implemented to cause the light source to change a visual property of the light when the processor system determines the presence of the interventional instrument based on the ultrasound.

Example 59 includes the ultrasound system as described in example 58, wherein the visual property of the light includes at least one of a color, a brightness, and a blinking pattern.

Example 60 includes the ultrasound system as described in example 54, wherein the ultrasound scanner includes a first light source and a second light source, wherein the processor system is implemented to cause the first light source to display light when the processor system determines a presence of the interventional instrument based on the additional ultrasound.

Example 61 includes the ultrasound system as described in example 60, wherein the processor system is implemented to cause the second light source to display additional light when the processor system determines the presence of the interventional instrument based on the additional ultrasound.

Example 62 includes the ultrasound system as described in example 61, wherein the processor system is implemented to cause the first light source to cease the display of the light when the processor system determines the presence of the interventional instrument based on the additional ultrasound.

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in some embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware (e.g., ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without other input or prompting, whether these features, elements or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, or Z). Thus, such disjunctive language is not generally intended to, and should not, imply that certain embodiments require at least one of X, at least one of Y, and at least one of Z to each be present.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

We claim:

1. An ultrasound system comprising:
a display device configured to display a user interface for the ultrasound system;
a wearable ultrasound scanner configured to be attached to a patient over a port that is placed inside the patient, the port configured to supply fluid to the patient or retrieve additional fluid from the patient; and
a processor system configured to:
generate, based on reflections of ultrasound received by the wearable ultrasound scanner, a health status of the port; and
cause the user interface to display the health status of the port.

2. The ultrasound system as described in claim 1, wherein the processor system is implemented to generate the health status of the port including a prediction of when the port will fail.

3. The ultrasound system as described in claim 1, further comprising a transceiver, wherein the processor system is implemented to cause the transmitter to communicate, over a network, the health status to the display device to cause the user interface to display the health status.

4. The ultrasound system as described in claim 3, wherein the processor system is implemented to:
determine, based on the health status of the port, transmission times;
generate additional health statuses of the port; and
communicate, at the transmission times, the additional health statuses over the network to at least one of the display device and a medical archiver.

5. The ultrasound system as described in claim 1, wherein the processor system is implemented to generate the health status of the port based on at least one of an indication of infection of tissue proximate to the port, an amount of swelling of the tissue, an indication of congestion in the port, a measure of volume flow of the fluid or the additional fluid, a measure of the temperature of the tissue proximate to the port and a measure of pressure of the fluid or the additional fluid.

6. The ultrasound system as described in claim 1, wherein the wearable ultrasound scanner includes a patch that includes the processor system and a coupling agent implemented to couple the ultrasound from the wearable ultrasound scanner to the patient and couple the reflections from the patient to the wearable ultrasound scanner.

7. The ultrasound system as described in claim 1, wherein the wearable ultrasound scanner includes a first transducer array and a second transducer array, the first transducer array implemented to transmit the ultrasound and the second transducer array implemented to receive the reflections of the ultrasound, and further wherein the first transducer array and the second transducer array are removably attached to the patch so that the first transducer array and the second transducer array can be removed and reattached to the patch at different positions on the patch.

8. The ultrasound system as described in claim 1, wherein the wearable ultrasound scanner includes a patch and the display device is removably attachable to the patch.

9. An ultrasound system comprising:
a wearable ultrasound scanner configured to be attached to a patient over a port that is placed inside the patient, the port configured to supply fluid to the patient or retrieve additional fluid from the patient, the wearable ultrasound scanner including insertion holes through which a needle can be inserted into the port for the supply of the fluid or the retrieval of the additional fluid; and
a processor system configured to:
generate, based on reflections of ultrasound received by the wearable ultrasound scanner, a health status of the port;
determine, based on the health status of the port, a recommended one of the insertion holes for the insertion of the needle; and
cause an indication of the recommended one of the insertion holes to be exposed.

10. The ultrasound system as described in claim 9, wherein the wearable ultrasound scanner includes light sources proximate to the insertion holes, and exposure of the indication of the recommended one of the insertion holes includes activating a light source of the light sources that is proximate to the recommended one of the insertion holes.

11. The ultrasound system as described in claim 9, further comprising a display device implemented to display the indication of the recommended one of the insertion holes, and further wherein the display device is implemented to display an orientation for the needle for the insertion through the recommended one of the insertion holes.

12. The ultrasound system as described in claim 9, wherein the processor system is implemented to generate the health status of the port based on at least one of an indication of infection of tissue proximate to the port, an amount of swelling of the tissue, an indication of congestion in the port, a measure of volume flow of the fluid or the additional fluid, a measure of the temperature of the tissue proximate to the port, and a measure of pressure of the fluid or the additional fluid.

13. The ultrasound system as described in claim 9, wherein the wearable ultrasound scanner includes multiple transducer arrays, and the processor system is implemented to:
determine one transducer array of the multiple transducer arrays that covers the current position of the tip of the needle; and
select, based on the one transducer array, a color or lighting pattern of the light source.

14. The ultrasound system as described in claim 9, wherein the processor system is implemented to:
determine, based on the health status of the port, an additional one of the insertion holes not recommended to use for the insertion of the needle; and
cause to be exposed an indication that the additional one of the insertion holes is not recommended for the insertion of the needle.

15. An ultrasound system comprising:
a display device configured to display guidance for placing a wearable ultrasound scanner over a port that is placed inside a patient, the port configured to supply fluid to the patient or retrieve additional fluid from the patient;
the wearable ultrasound scanner; and
a processor system configured to:
generate, based on reflections of ultrasound received by the wearable ultrasound scanner, a health status of the port; and
generate, based on the health status of the port, the guidance for placement of the wearable ultrasound scanner.

16. The ultrasound system as described in claim 15, wherein the processor system is implemented to generate the health status of the port based on at least one of an indication of infection of tissue proximate to the port, an amount of swelling of the tissue, an indication of congestion in the port, a measure of volume flow of the fluid or the additional fluid, and a measure of pressure of the fluid or the additional fluid.

17. The ultrasound system as described in claim 15, further comprising a transceiver implemented to transmit the health status of the port to a nurse station of a care facility.

18. The ultrasound system as described in claim 15, wherein the fluid includes at least one of a drug, saline fluid, dextrose fluid, lactated Ringer's fluid, and blood.

19. The ultrasound system as described in claim 15, wherein the additional fluid includes at least one of blood, urine, extracellular fluid, semen, amniotic fluid, cerebrospinal fluid, and plasma.

20. The ultrasound system as described in claim 15, wherein the guidance includes a location and an orientation of the wearable ultrasound scanner, the location and the orientation being relative to the port.

21. The ultrasound system as described in claim 20, wherein the wearable ultrasound scanner includes multiple transducer arrays, and the processor system is implemented to:
select, based on the health status of the port, one of the multiple transducer arrays; and
determine at least one of the location and the orientation so that the one of the multiple transducer arrays is positioned over the port.

22. The ultrasound system as described in claim 15, wherein the processor system is implemented to, after the wearable ultrasound scanner is placed on the patient based on the guidance, generate, based on additional reflections of ultrasound received by the wearable ultrasound scanner, an additional health status of the port, and further wherein the additional health status of the port includes an expected time to failure for the port.
